SEQUENCE LISTING (22 sequences)

SEQ ID NO: 1 - protein, 5 aa, synthetic construct
SYNMH

SEQ ID NO: 2 - protein, 15 aa, synthetic construct
AIYPGNGDTSYNQKF

SEQ ID NO: 3 - protein, 13 aa, synthetic construct
VVYYSNSYWYFDV

SEQ ID NO: 4 - protein, 10 aa, synthetic construct
RASSSVSYMH

SEQ ID NO: 5 - protein, 7 aa, synthetic construct
APSNLAS

SEQ ID NO: 6 - protein, 9 aa, synthetic construct
QQWSFNPPT

SEQ ID NO: 7 - protein, 122 aa, synthetic construct
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSS

SEQ ID NO: 8 - protein, 122 aa, synthetic construct [sequence omitted]

SEQ ID NO: 9 - protein, 452 aa, synthetic construct
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 452 aa, synthetic construct
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 452 aa, synthetic construct
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 452 aa, synthetic construct
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCAVSNKALPAPIEATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 452 aa, synthetic construct
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 107 aa, synthetic construct
QIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKR

SEQ ID NO: 15 - protein, 107 aa, synthetic construct
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKR

SEQ ID NO: 16 - protein, 213 aa, synthetic construct
DIQMTQSPSSLSASVGDRVTITCRASSSVSYLHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 213 aa, synthetic construct
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 297 aa, synthetic construct
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 19 - protein, 10 aa, synthetic construct
GYTFTSYNMH

SEQ ID NO: 20 - protein, 10 aa, synthetic construct
AIYPGNGDTS

SEQ ID NO: 21 - protein, 7 aa, synthetic construct
GYTFTSY

SEQ ID NO: 22 - protein, 6 aa, synthetic construct
YPGNGD

(19) United States
(12) Patent Application Publication
(10) Pub. No.: US 2025/0179207 A1
(43) Pub. Date: Jun. 5, 2025

(54) METHODS FOR TREATING DIGITALLY-IDENTIFIED CD20-RELATED DISORDERS

(71) Applicant: Genzyme Corporation, Cambridge, MA (US)

(72) Inventor: Cliona Marie Molony, Boston, MA (US)

(21) Appl. No.: 18/960,796

(22) Filed: Nov. 26, 2024

Related U.S. Application Data
(60) Provisional application No. 63/604,675, filed on Nov. 30, 2023.

(51) Int. Cl. C07K 16/28; G16H 50/20

(52) U.S. Cl. CPC C07K 16/2887; G16H 50/20; C07K 2317/565

(57) ABSTRACT

Disclosed are methods of treating various disorders with anti-CD20 antibodies and fragments thereof.

### CROSS-REFERENCE TO RELATED APPLICATION

This application claims priority to U.S. Application No. 63/604,675, filed on Nov. 30, 2023. The contents of this application are incorporated by reference in its entirety.

### SEQUENCE LISTING

This application contains a Sequence Listing that has been submitted electronically as an XML file named “37488-0795001_ST26_SL.XML” The XML file, created on Nov. 25, 2024, is 23,411 bytes in size. The material in the XML file is hereby incorporated by reference in its entirety.

### TECHNICAL FIELD

The present disclosure relates to treating various disorders with an anti-CD20 antibody.

### BACKGROUND

Lymphocytes are one of several populations of white blood cells; they specifically recognize and respond to foreign antigen. The three major classes of lymphocytes are B lymphocytes (B cells), T lymphocytes (T cells) and natural killer (NK) cells. B lymphocytes are the cells responsible for antibody production and provide humoral immunity. B cells mature within the bone marrow and leave the marrow expressing an antigen-binding antibody on their cell surface. When a naive B cell first encounters the antigen for which its membrane-bound antibody is specific, the cell begins to divide rapidly and its progeny differentiate into memory B cells and effector cells called “plasma cells”. Memory B cells have a longer life span and continue to express membrane-bound antibody with the same specificity as the original parent cell. Plasma cells do not produce membrane-bound antibody but instead produce secreted form of the antibody. Secreted antibodies are the major effector molecules of humoral immunity.

The CD20 antigen (also called human B-lymphocyte-restricted differentiation antigen, Bp35) is a hydrophobic transmembrane protein with a molecular weight of approximately 35 kD located on pre-B and mature B lymphocytes (Valentine et al. J. Biol. Chem. 264(19):11282-11287 (1989); and Einfeld et al. EMBO J. 7(3):711-717 (1988)). The antigen is also expressed on greater than 90% of B cell non-Hodgkin's lymphomas (NHL) (Anderson et al. Blood 63(6):1424-1433 (1984)), but is not found on hematopoietic stem cells, pro-B cells, normal plasma cells or other normal tissues (Tedder et al. J. Immunol. 135(2):973-979 (1985)). CD20 is thought to regulate an early step(s) in the activation process for cell cycle initiation and differentiation (Tedder et al., supra) and possibly functions as a calcium ion channel (Tedder et al. J. Cell. Biochem. 14D:195 (1990)).

Given the expression of CD20 in B cell lymphomas, this antigen has been a useful therapeutic target to treat such lymphomas. There are more than 300,000 people in the United States with B-cell NHL and more than 56,000 new cases are diagnosed each year. For example, the rituximab (RITUXAN®) antibody which is a genetically engineered chimeric murine/human monoclonal antibody directed against human CD20 antigen (commercially available from Genentech, Inc., South San Francisco, Calif, U.S.) is used for the treatment of patients with relapsed or refractory low-grade or follicular, CD20 positive, B cell non-Hodgkin's lymphoma. Rituximab is the antibody referred to as “C2B8” in U.S. Pat. No. 5,736,137 issued Apr. 7, 1998 (Anderson et al.) and in U.S. Pat. No. 5,776,456. In vitro mechanism of action studies have demonstrated that RITUXAN® (rituximab) binds human complement and lyses lymphoid B cell lines through complement-dependent cytotoxicity (CDC) (Reff et al. Blood 83(2):435-445 (1994)). Additionally, it has significant activity in assays for antibody-dependent cellular cytotoxicity (ADCC). In vivo preclinical studies have shown that RITUXAN® (rituximab) depletes B cells from the peripheral blood, lymph nodes, and bone marrow of cynomolgus monkeys, presumably through complement and cell-mediated processes (Reff et al. Blood 83(2):435-445 (1994)). Other anti-CD20 antibodies indicated for the treatment of NHL include the murine antibody Zevalin™ (Ibritumomab Tiuxetan) which is linked to the radioisotope, Yttrium-90 (IDEC Pharmaceuticals, San Diego, Calif.), Bexxar™ (Tositumomab and Iodine|131 Tositumomab) which is a another fully murine antibody conjugated to I-131 (Corixa, Wash.).

A major limitation in the use of murine antibodies in human therapy is the human anti-mouse antibody (HAMA) response (see, e.g., Miller, R. A. et al. “Monoclonal antibody therapeutic trials in seven patients with T-cell lymphoma” Blood, 62:988-995, 1983; and Schroff, R. W., et al. “Human anti-murine immunoglobulin response in patients receiving monoclonal antibody therapy” Cancer Res., 45:879-885, 1985). Even chimeric molecules, where the variable (V) domains of rodent antibodies are fused to human constant (C) regions, are still capable of eliciting a significant immune response (HACA, human anti-chimeric antibody) (Neuberger et al. Nature (Lond.), 314:268-270, 1985). A powerful approach to overcome these limitations in the clinical use of monoclonal antibodies is “humanization” of the murine antibody or antibody from a non-human species (Jones et al. Nature (Lond), 321:522-525, 1986; Riechman et al., Nature (Lond), 332:323-327, 1988).

There remains a need to use an anti-CD20 antibody to treat other disorders.

### SUMMARY

The present disclosure is based, at least in part, on the discovery of new disorders treatable with an anti-CD20 antibody. Treatable disorders described herein were identified at least initially using a novel computational method capable of analyzing large volumes of patient population data and predicting the therapeutic outcome of treatment using the anti-4Rα antibody.

Therefore, disclosed herein is a method of treating a subject with an anti-CD20 antibody, the method comprising administering a therapeutically effective amount of the anti-CD20 antibody to a subject exhibiting at least one symptom of, or determined to be susceptible to, a CD20-related disorder selected from the group consisting of vascular myelopathy, ankylosing spondylitis, scleroderma, narcolepsy, gastro malabsorption conditions, complex regional pain syndrome, a cluster headache, amyloidosis, cystitis, aseptic necrosis, or combinations thereof, wherein the anti-CD20 antibody comprises: a variable heavy chain CDR1 of SEQ ID NO:1 (SYNMH); a variable heavy chain CDR2 of SEQ ID NO:2 (AIYPGNGDTSYNQKF); a variable heavy chain CDR3 of SEQ ID NO:3 (VVYYSNSYWYFDV); a variable light chain CDR1 of SEQ ID NO:4 (RASSSVSYMH); a variable light chain CDR2 of SEQ ID NO:5 (APSNLAS); and a variable light chain CDR3 of SEQ ID NO:6 (QQWSFNPPT).

Also disclosed herein is a method of treating a subject with an anti-CD20 antibody, the method comprising administering a therapeutically effective amount of the anti-CD20 antibody to a subject exhibiting at least one symptom of, or determined to be susceptible to, a CD20-related disorder selected from the group consisting of vascular myelopathy, ankylosing spondylitis, scleroderma, narcolepsy, gastro malabsorption conditions, complex regional pain syndrome, a cluster headache, amyloidosis, cystitis, aseptic necrosis, or combinations thereof, wherein the anti-CD20 antibody or antigen-binding fragment useful in the presently described methods includes three HCDRs (HCDR1, HCDR2 and HCDR3) and three LCDRs (LCDR1, LCDR2 and LCDR3), wherein the HCDR1 includes the amino acid sequence of SEQ ID NO:19 (GYTFTSYNMH); the HCDR2 includes the amino acid sequence of SEQ ID NO:20 (AIYPGNGDTS); the HCDR3 includes the amino acid sequence of SEQ ID NO:3 (VVYYSNSYWYFDV); the LCDR1 includes the amino acid sequence of SEQ ID NO:4 (RASSSVSYMH); the LCDR2 includes the amino acid sequence of SEQ ID NO:5 (APSNLAS); and the LCDR3 includes the amino acid sequence of SEQ ID NO:6 (QQWSFNPPT).

Also disclosed herein is a method of treating a subject with an anti-CD20 antibody, the method comprising administering a therapeutically effective amount of the anti-CD20 antibody to a subject exhibiting at least one symptom of, or determined to be susceptible to, a CD20-related disorder selected from the group consisting of vascular myelopathy, ankylosing spondylitis, scleroderma, narcolepsy, gastro malabsorption conditions, complex regional pain syndrome, a cluster headache, amyloidosis, cystitis, aseptic necrosis, or combinations thereof, wherein the anti-CD20 antibody or antigen-binding fragment useful in the presently described methods includes three HCDRs (HCDR1, HCDR2 and HCDR3) and three LCDRs (LCDR1, LCDR2 and LCDR3), wherein the HCDR1 includes the amino acid sequence of SEQ ID NO:21 (GYTFTSY); the HCDR2 includes the amino acid sequence of SEQ ID NO:22 (YPGNGD); the HCDR3 includes the amino acid sequence of SEQ ID NO:3 (VVYYSNSYWYFDV); the LCDR1 includes the amino acid sequence of SEQ ID NO:4 (RASSSVSYMH); the LCDR2 includes the amino acid sequence of SEQ ID NO:5 (APSNLAS); and the LCDR3 includes the amino acid sequence of SEQ ID NO:6 (QQWSFNPPT).

Also disclosed herein is a method of treating a subject exhibiting at least one symptom of, or determined to be susceptible to, a CD20-related disorder with an anti-CD20 antibody, the method comprising: (a) selecting a reference characteristic or a plurality of reference characteristics related to a CD20 pathway in a set of data representing medical records of a plurality of reference subjects, wherein each reference subject in the plurality of reference subjects has one or more reference CD20-related disorders; (b) clustering, by a computer system, in accordance with the characteristic or the plurality of characteristics related to the CD20 pathway, a subset of reference subjects from the plurality of reference subjects, wherein the subset comprises the characteristic or the plurality of characteristics; (c) identifying in the subset clustered in step (b) one or more CD20 features, wherein the one or more CD20 features is different from the reference characteristic or the plurality of reference characteristics; (d) selecting the subject exhibiting the one or more CD20 features, thereby identifying the subject as having at least one symptom of, or determined to be susceptible to, the CD20-related disorder; and (e) administering a therapeutically effective amount of the anti-CD20 antibody to the subject, wherein the anti-CD20 antibody comprises: a variable heavy chain CDR1 of SEQ ID NO:1 (SYNMH); a variable heavy chain CDR2 of SEQ ID NO:2 (AIYPGNGDTSYNQKF); a variable heavy chain CDR3 of SEQ ID NO:3 (VVYYSNSYWYFDV); a variable light chain CDR1 of SEQ ID NO:4 (RASSSVSYMH); a variable light chain CDR2 of SEQ ID NO:5 (APSNLAS); and a variable light chain CDR3 of SEQ ID NO:6 (QQWSFNPPT), thereby treating the subject.

In some instances, the reference characteristic or plurality of reference characteristics comprises having received treatment of at least one dose of the anti-CD20 antibody. In some instances, the reference characteristic or plurality of reference characteristics having received treatment of two or more doses of the anti-CD20 antibody. In some instances, the reference characteristic or plurality of reference characteristics comprises having received prior treatment of at least one dose of rituximab, ofatumumab, or ublituximab. In some instances, the reference characteristic or plurality of reference characteristics having received prior treatment of two or more doses of rituximab, ofatumumab, or ublituximab.

In some instances, the one or more reference CD20-related disorders comprises multiple sclerosis. In some instances, the multiple sclerosis is primary progressive multiple sclerosis (PPMS). In some instances, the multiple sclerosis is a relapsing form of multiple sclerosis. In some instances, the multiple sclerosis is secondary progressive multiple sclerosis (PPMS). In some instances, the multiple sclerosis is relapsing remitting multiple sclerosis (RRMS). In some instances, the one or more reference CD20-related disorders is selected from clinically isolated syndrome, non-Hodgkin's lymphoma (NHL), chronic lymphocytic leukemia (CLL), rheumatoid arthritis (RA), granulomatosis with polyangiitis (GPA), microscopic polyangiitis (MPA), pemphigus vulgaris (PV), follicular lymphoma, or combinations thereof.

In some instances, the CD20-related disorder is selected from a vascular myelopathy, ankylosing spondylitis, scleroderma, narcolepsy, gastro malabsorption conditions, complex regional pain syndrome, a cluster headache, and combinations thereof. In some instances, the CD20-related disorder is selected from amyloidosis, cystitis, aseptic necrosis, and combinations thereof.

In some instances, the clustering step is performed at least two, at least three, at least four or more times.

In some instances, the anti-CD20 antibody comprises: (a) a heavy chain variable region (VH) comprising the amino acid sequence selected from:

(SEQ ID NO: 7)
QAYLQQSGAELVRPGASVKMSCKASGYTFTSYNMHWVKQTPRQGL
EWIGAIYPGNGDTSYNQKFKGKATLTVDKSSSTAYMQLSSLTSED
SAVYFCARVVYYSNSYWYFDVWGTGTTVTVSS;
or
(SEQ ID NO: 8)
EVQLVESGGGLVQPGGSLRLSCAASGYTFTSYNMHWVRQAPGKGL
EWVGAIYPGNGDTSYNQKFKGRFTISVDKSKNTLYLQMNSLRAED
TAVYYCARVVYYSNSYWYFDVWGQGTLVTVSS;
and
(b) a light chain variable region (VL) comprising
the amino acid sequence selected from:
(SEQ ID NO: 9)
QIVLSQSPAILSASPGEKVTMTCRASSSVSYMHWYQQKPGSSPKP
WIYAPSNLASGVPARFSGSGSGTSYSLTISRVEAEDAATYYCQQW
SFNPPTFGAGTKLELKR;
or
(SEQ ID NO: 10)
DIQMTQSPSSLSASVGDRVTITCRASSSVSYMHWYQQKPGKAPKP
LIYAPSNLASGVPSRFSGSGSGTDFTLTISSLQPEDFATYYCQQW
SFNPPTFGQGTKVEIKR.

In some instances, the anti-CD20 antibody comprises: (a) a heavy chain sequence comprising:

(SEQ ID NO: 11)
EVQLVESGGGLVQPGGSLRLSCAASGYTFTSYNMHWVRQAPGKGL
EWVGAIYPGNGDTSYNQKFKGRFTISVDKSKNTLYLQMNSLRAED
TAVYYCARVVYYSNSYWYFDVWGQGTLVTVSSASTKGPSVFPLAP
SSKSTSGGTAALGCLVKDYFPEPVTVSWNSGALTSGVHTFPAVLQ
SSGLYSLSSVVTVPSSSLGTQTYICNVNHKPSNTKVDKKVEPKSC
DKTHTCPPCPAPELLGGPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
EEMTKNQVSLTCLVKGFYPSDIAVEWESNGQPENNYKTTPPVLDS
DGSFFLYSKLTVDKSRWQQGNVFSCSVMHEALHNHYTQKSLSLSP
GK;
and
(b) a light chain sequence comprising:
(SEQ ID NO: 12)
DIQMTQSPSSLSASVGDRVTITCRASSSVSYMHWYQQKPGKAPKP
LIYAPSNLASGVPSRFSGSGSGTDFTLTISSLQPEDFATYYCQQW
SFNPPTFGQGTKVEIKRTVAAPSVFIFPPSDEQLKSGTASVVCLL
NNFYPREAKVQWKVDNALQSGNSQESVTEQDSKDSTYSLSSTLTL
SKADYEKHKVYACEVTHQGLSSPVTKSFNRGEC.

In some instances, the anti-CD20 antibody is in a pharmaceutical composition, wherein the pharmaceutical composition comprises a pharmaceutically acceptable carrier.

In some instances, the anti-CD20 antibody is selected from a Fab fragment, a F(ab′)2 fragment, a scFv, and a scAb. In some instances, the subject is human. In some instances, the administering is intradermal, intramuscular, intraperitoneal, intravenous, subcutaneous, intranasal, or epidural administration. In some instances, the administering is intravenous. In some instances, the anti-CD20 antibody is administered over multiple doses. In some instances, the anti-CD20 antibody is administered over two doses. In some instances, the subject has never been previously treated with the anti-CD20 antibody. In some instances, the anti-CD20 antibody is administered at a dose of about 0.0001 to about 10 mg/kg of subject body weight. In some instances, the method also includes administering a second therapeutic agent. In some instances, the second therapeutic agent is a second antibody or antigen binding fragment thereof, a soluble cytokine receptor, an IgE antagonist, an anti-asthma medication, or a checkpoint inhibitor. In some instances, the anti-asthma medication is corticosteroids, non-steroidal agents, beta agonists, leukotriene antagonists, xanthines, fluticasone, salmeterol, or albuterol. In some instances, the checkpoint inhibitor is a PD-1 antagonist, a PD-L1 antagonist, or a CTLA-4 antagonist. In some instances, the anti-CD20 antibody is administered prior to administering the second therapeutic agent. In some instances, the anti-CD20 antibody is administered concurrently with the administering of the second therapeutic agent. In some instances, the anti-CD20 antibody is administered subsequent to the administering of the second therapeutic agent.

In some instances, disclosed are uses of a compositions comprising an anti-CD20 (e.g., Ocrevus®) in the manufacture of a medicament for the treatment of any of the indications provided herein. In some instances, the indications include vascular myelopathy, ankylosing spondylitis, scleroderma, narcolepsy, gastro malabsorption conditions, complex regional pain syndrome, a cluster headache, amyloidosis, cystitis, aseptic necrosis, or combinations thereof. In some instances, the anti-CD20 antibody comprises: a variable heavy chain CDR1 of SEQ ID NO:1 (SYNMH); a variable heavy chain CDR2 of SEQ ID NO:2 (AIYPGNGDTSYNQKF); a variable heavy chain CDR3 of SEQ ID NO:3 (VVYYSNSYWYFDV); a variable light chain CDR1 of SEQ ID NO:4 (RASSSVSYMH); a variable light chain CDR2 of SEQ ID NO:5 (APSNLAS); and a variable light chain CDR3 of SEQ ID NO:6 (QQWSFNPPT). In some instances, the an anti-CD20 antibody or antigen-binding fragment useful in the presently described methods includes three HCDRs (HCDR1, HCDR2 and HCDR3) and three LCDRs (LCDR1, LCDR2 and LCDR3), wherein the HCDR1 includes the amino acid sequence of SEQ ID NO:19 (GYTFTSYNMH); the HCDR2 includes the amino acid sequence of SEQ ID NO:20 (AIYPGNGDTS); the HCDR3 includes the amino acid sequence of SEQ ID NO:3 (VVYYSNSYWYFDV); the LCDR1 includes the amino acid sequence of SEQ ID NO:4 (RASSSVSYMH); the LCDR2 includes the amino acid sequence of SEQ ID NO:5 (APSNLAS); and the LCDR3 includes the amino acid sequence of SEQ ID NO:6 (QQWSFNPPT). In some instances, the anti-CD20 antibody or antigen-binding fragment useful in the presently described methods includes three HCDRs (HCDR1, HCDR2 and HCDR3) and three LCDRs (LCDR1, LCDR2 and LCDR3), wherein the HCDR1 includes the amino acid sequence of SEQ ID NO:21 (GYTFTSY); the HCDR2 includes the amino acid sequence of SEQ ID NO:22 (YPGNGD); the HCDR3 includes the amino acid sequence of SEQ ID NO:3 (VVYYSNSYWYFDV); the LCDR1 includes the amino acid sequence of SEQ ID NO:4 (RASSSVSYMH); the LCDR2 includes the amino acid sequence of SEQ ID NO:5 (APSNLAS); and the LCDR3 includes the amino acid sequence of SEQ ID NO:6 (QQWSFNPPT).

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various aspects of the features of this disclosure are described herein. However, it should be understood that such aspects are provided merely by way of example, and numerous variations, changes, and substitutions can occur to those skilled in the art without departing from the scope of this disclosure. It should also be understood that various alternatives to the specific aspects described herein are also within the scope of this disclosure.

### BRIEF DESCRIPTION OF DRAWINGS

FIG. 1 is a diagram illustrating an example of a data processing system for identification of indications for treatment with a drug.

FIG. 2 is a flow chart illustrating an example method for identifying indications for treatment with a drug.

FIG. 3 is a diagram illustrating an experiment using the systems and methods described herein.

FIG. 4 is a further diagram illustrating other aspects of the systems and methods described in this specification, including how certain anchor indications were defined and used to identify a patient cohort, features, and referential indications, as well as other aspects of feature selection, clustering, and indication ranking.

FIG. 5 is a block diagram of an example computer system used to provide computational functionalities associated with described algorithms, methods, functions, processes, flows, and procedures described in the present disclosure.

FIG. 6 shows enriched indications after performing the methods disclosed herein.

### DETAILED DESCRIPTION

The present disclosure is based, at least in part, on the discovery that an anti-CD20 antibody can be used to treat various disorders in addition to those currently approved by the FDA. FDA-approved anti-CD20 drugs include, but are not limited to, Ocrevus® (generic name: ocrelizumab); Rituxan (rituximab), Kesimpta (ofatumumab), Gazyva (obinutuzumab), Zevalin (ibritumomab), Y-90 Zevalin (ibritumomab), Truxima, Ruxience, Rituxan Hycela (hyaluronidase/rituximab), Riabni, In-111 Zevalin (ibritumomab), Briumvi (ublituximab) Bexxar (iodine i 131 tositumomab), and Arzerra (ofatumumab).

Ocrevus® (ocrelizumab) is a therapeutic monoclonal antibody is a humanized monoclonal antibody designed to selectively target CD20-positive B cells. Ocrevus® is indicated as a treatment for both relapsing (RMS) and primary progressive (PPMS) forms of multiple sclerosis. In some instances, Ocrevus® is given once every six months by an intravenous (IV) infusion.

The new indications, which are the subject of the present specification, were at least in part identified using computational methods that analyze data obtained from millions of patients and predict therapeutic outcomes for specific drugs. Starting with those predictions, the present inventors have developed treatments for various diseases and disorders described herein using an anti-CD20 antibody (e.g., Ocrevus®).

As used herein, the “CD20” antigen is a non-glycosylated, transmembrane phosphoprotein with a molecular weight of approximately 35 kD that is found on the surface of greater than 90% of B cells from peripheral blood or lymphoid organs. CD20 is expressed during early pre-B cell development and remains until plasma cell differentiation; it is not found on human stem cells, lymphoid progenitor cells or normal plasma cells. CD20 is present on both normal B cells as well as malignant B cells. Other names for CD20 in the literature include “B-lymphocyte-restricted differentiation antigen” and “Bp35”. The CD20 antigen is described in, for example, Clark and Ledbetter, Adv. Can. Res. 52:81-149 (1989) and Valentine et al. J. Biol. Chem. 264(19):11282-11287 (1989).

The term “antibody”, as used herein, refers to immunoglobulin molecules including four polypeptide chains, two heavy (H) chains and two light (L) chains inter-connected by disulfide bonds. Each heavy chain includes a heavy chain variable region (HCVR or VH) and a heavy chain constant region. The heavy chain constant region includes three domains, CH1, CH2 and CH3. Each light chain includes a light chain variable region (LCVR or VL) and a light chain constant region. The light chain constant region includes one domain (CL1). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

The term “antigen-binding fragment” of an antibody (alternatively referred to as “antigen-binding portion” or “antibody fragment”) refers to a fragment of an antibody that retains an ability to specifically bind to an antigen. It has been shown that the antigen-binding function of an antibody can be performed by fragments of a full-length antibody Exemplary antigen-binding fragments include (i) a Fab fragment, a monovalent fragment consisting of the VL, VH, CL1 and CH1 domains; (ii) a F(ab′)2 fragment, a bivalent fragment including two F(ab)′ fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) Nature 241:544-546), which consists of a VH domain; and (vi) a CDR. Furthermore, although the two domains of the Fv fragment, VL and VH, are coded for by separate genes, they can be joined, using recombinant methods, by a synthetic linker that enables them to be made as a single contiguous chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883. Such single chain antibodies are also encompassed within the term “antigen-binding fragment” of an antibody. Other forms of single chain antibodies, such as diabodies, are also encompassed (see e.g., Holliger et al. (1993) Proc. Natl. Acad Sci. USA 90:6444-6448). A more detailed description of antigen-binding fragments useful in the present disclosure is provided below.

A “neutralizing” or “blocking” antibody, refers to an antibody whose binding to human CD20 (hCD20) results in inhibition of the biological activity of an antigen to CD20.

A “CDR” or “complementarity determining region” is a region of hypervariability interspersed within regions that are more conserved, termed “framework regions” (FR). In different embodiments of the anti-hCD20 antibody or antigen-binding fragment of the disclosure, the FRs can be identical to the human germline sequences, or can be naturally or artificially modified.

The term “epitope” is an antigenic determinant that interacts with a specific antigen binding site in the variable region of an antibody molecule known as a paratope. A single antigen can have more than one epitope. Epitopes can be either conformational or linear. A conformational epitope is produced by spatially juxtaposed amino acids from different segments of the linear polypeptide chain. A linear epitope is one produced by adjacent amino acid residues in a polypeptide chain. In certain circumstances, an epitope can include moieties of saccharides, phosphoryl groups, or sufonyl groups on the antigen.

The terms “substantial identity,” “substantially identical,” “substantial similarity,” “substantially similar” derivatives and variations when referring to a nucleic acid or protein refers to sequences that are at least about 75% identical to the SEQ ID NOs: 1-16, described herein, can be used in the methods and compositions described herein. In some instances, the nucleotide sequences are about 80%, 85%, 90%, 95%, 99% or 100% identical.

To determine the percent identity of two sequences, the sequences are aligned for optimal comparison purposes (gaps are introduced in one or both of a first and a second amino acid or nucleic acid sequence as required for optimal alignment, and non-homologous sequences can be disregarded for comparison purposes). The length of a reference sequence aligned for comparison purposes is at least 80% (in some embodiments, about 85%, 90%, 95%, or 100% of the length of the reference sequence) is aligned. The nucleotides or residues at corresponding positions are then compared. When a position in the first sequence is occupied by the same nucleotide or residue as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J. Mol. Biol. 48:444-453) algorithm which has been incorporated into the GAP program in the GCG software.

As used herein, the terms “subject” and “patient” are used interchangeably. The subject can be an animal. In some instances, the subject is a mammal such as a non-primate (e.g., cow, pig, horse, cat, dog, rat, etc.) or a primate (e.g., monkey or human). In some instances, the subject is a human. In certain instances, such terms refer to a non-human animal (e.g., a non-human animal such as a pig, horse, cow, cat, or dog).

As used herein, the terms “about” and “approximately,” when used to modify a numeric value or numeric range, indicate that deviations of 5% to 10% above and 5% to 10% below the value or range remain within the intended meaning of the recited value or range.

It is understood that wherever aspects are described herein with the language “comprising,” otherwise analogous aspects described in terms of “consisting of” and/or “consisting essentially of” are also provided.

The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

### Anti-CD20 Antibodies, Antigen-Binding Fragments, and Compositions

The present disclosure describes, inter alia, the use of anti-CD20 antibodies to treat various disorders. The various disorders were identified as being treatable with anti-CD20 antibodies using the digital process of analyzing patient data described herein. Prior to the present disclosure, these disorders were not known to be treatable with an anti-CD20 such as Ocrevus®. Accordingly, a brief description of anti-CD20 antibodies and fragments, compositions, and dosages, useful in the presently-described methods is provided below.

### I. Antibodies and Fragments

Of particular use in the present disclosure are anti-CD20 antibodies. Skilled practitioners will appreciate that a complete antibody includes four polypeptide chains, two heavy (H) chains and two light (L) chains, inter-connected by disulfide bonds, and in some instances includes multimers thereof (e.g., IgM). In a typical antibody, each heavy chain includes a heavy chain variable region (abbreviated herein as HCVR or VH) and a heavy chain constant region. The heavy chain constant region includes three domains, CH1, CH2 and CH3. Each light chain includes a light chain variable region (abbreviated herein as LCVR or VL) and a light chain constant region. The light chain constant region includes one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In different embodiments of the disclosure, the FRs of the anti-CD20 antibody (or antigen-binding portion thereof) can be identical to the human germline sequences, or can be naturally or artificially modified. An amino acid consensus sequence can be defined based on a side-by-side analysis of two or more CDRs.

An antigen-binding fragment of a full antibody can also be useful in the presently-described methods. An antigen-binding fragment (e.g., of an anti-CD20 antibody) can be, e.g., any naturally occurring, enzymatically obtainable, synthetic, or genetically engineered polypeptide or glycoprotein that specifically binds an antigen to form a complex. Antigen-binding fragments of an antibody can be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA can be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody typically includes at least one variable domain. The variable domain can be of any size or amino acid composition and generally includes at least one CDR which is adjacent to or in frame with one or more framework sequences. In antigen-binding fragments having a VH domain associated with a VL domain, the VH and VL domains can be situated relative to one another in any suitable arrangement. For example, the variable region can be dimeric and contain VH-VH, VH-VL or VL-VL dimers.

Alternatively, the antigen-binding fragment of an antibody can contain a monomeric VH or VL domain.

In certain instances, an antigen-binding fragment of an antibody can include at least one variable domain covalently linked to at least one constant domain. Non-limiting, exemplary configurations of variable and constant domains that can be found within an antigen-binding fragment of an antibody include: (i) VH-CH1; (ii) VH-CH2; (iii) VH-CH3; (iv) VH-CH1-CH2; (v) VH-CH1-CH2-CH3; (vi) VH-CH2-CH3; (vii) VH-CL; (viii) VL-CH1; (ix) VL-CH2; (x) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed above, the variable and constant domains can be either directly linked to one another or can be linked by a full or partial hinge or linker region. A hinge region can consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure can include a homodimer or heterodimer (or other multimer) of any of the variable and constant domain configurations listed above in non-covalent association with one another and/or with one or more monomeric VH or VL domain (e.g., by disulfide bond(s)).

In some instances, an anti-CD20 antibody can be a multispecific (e.g., bispecific) antibody. A multispecific antibody or antigen-binding fragment of an antibody typically includes at least two different variable domains, wherein each variable domain is capable of specifically binding to a separate antigen or to a different epitope on the same antigen. Any multispecific antibody format can be adapted for use in the context of an antibody or antigen-binding fragment of an antibody of the present disclosure using routine techniques available in the art. For example, the present disclosure includes methods including the use of bispecific antibodies wherein one arm of an immunoglobulin is specific for CD20 or a fragment thereof, and the other arm of the immunoglobulin is specific for a second therapeutic target or is conjugated to a therapeutic moiety. Exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, and Mab2 bispecific formats (see, e.g., Klein et al. 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc. [Epub: Dec. 4, 2012]).

The antibodies used in the methods of the present disclosure can be human antibodies. The term “human antibody,” as used herein, is intended to include antibodies having variable and constant regions derived from human germline immunoglobulin sequences. The human antibodies of the disclosure can nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

Antibodies useful in the methods of the present disclosure can be recombinant human antibodies. The term “recombinant human antibody,” as used herein, is intended to include human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain instances, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, cannot naturally exist within the human antibody germline repertoire in vivo.

In some instances, antibodies used in the methods of the present disclosure specifically bind CD20. The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” CD20, as used in the context of the present disclosure, includes antibodies that bind CD20 or portion thereof with a Kd of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human CD20 can, however, have cross-reactivity to other antigens, such as CD20 molecules from other (non-human) species.

The term “variable” refers to the fact that certain portions of the variable regions of antibodies differ extensively in sequence among antibodies and are used in the binding and specificity of each particular antibody for its particular antigen. However, the variability is not evenly distributed throughout the variable regions of antibodies. The variability is concentrated in three segments called complementarity-determining regions (CDRs) or hypervariable regions both in the light-chain and the heavy-chain variable regions. The more highly conserved portions of variable regions are called the framework (FR). The variable regions of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen-binding site of antibodies. The constant domains are not involved directly in binding an antibody to an antigen, but exhibit various effector functions, such as participation of the antibody in antibody-dependent cellular toxicity. There are at least two techniques for determining CDRs: (1) an approach based on cross-species sequence variability (i.e., Kabat et al., Sequences of Proteins of Immunological Interest, (5th ed., 1991, National Institutes of Health, Bethesda Md.)); and (2) an approach based on crystallographic studies of antigen-antibody complexes (Al-lazikani et al., J. Molec. Biol. 273:927-948 (1997)). In addition, combinations of these two approaches are sometimes used in the art to determine CDRs.

The Kabat numbering system is generally used when referring to a residue in the variable region (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)).

The amino acid position numbering as in Kabat, refers to the numbering system used for heavy chain variable regions or light chain variable regions of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence can contain fewer or additional amino acids corresponding to a shortening of, or insertion into, a FR or CDR of the variable region. For example, a heavy chain variable region can include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues can be determined for a given antibody by alignment at regions of homology of the sequence of the antibody with a “standard” Kabat numbered sequence. Chothia refers instead to the location of the structural loops (Chothia et al., J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

The amino acid position numbering as in Kabat, refers to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence can contain fewer or additional amino acids corresponding to a shortening of, or insertion into, a FR or CDR of the variable domain. For example, a heavy chain variable domain can include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues can be determined for a given antibody by alignment at regions of homology of the sequence of the antibody with a “standard” Kabat numbered sequence. Chothia refers instead to the location of the structural loops (Chothia and Lesk, J. Mol. Biol. 196:901-917 (1987)), wherein the loops are also identified as light and heavy chain CDRs (e.g., L1=LC CDR1, etc.). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

TABLE 1
Amino Acid Position Numbering Systems

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |

(Kabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H95-H102 | H95-H102 | H95-H102 |

An Fc region as used herein includes the polypeptides comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. Although the boundaries of the Fc region may vary, the human IgG heavy chain Fc region is usually defined to comprise residues C226 or P230 to its carboxyl-terminus, wherein the numbering is according to the EU index as in Kabat et al., (1991, NIH Publication 91-3242, National Technical Information Service, Springfield, Va.). The “EU index as set forth in Kabat” refers to the residue numbering of the human IgG1 EU antibody as described in Kabat et al., supra. Fc may refer to this region in isolation, or this region in the context of an antibody, antibody fragment, or Fc fusion protein. An Fc variant protein may be an antibody, Fc fusion, or any protein or protein domain that comprises an Fc region. Particular proteins comprise variant Fc regions, which are non-naturally occurring variants of an Fc. Polymorphisms have been observed at a number of Fc positions, including, but not limited to, Kabat 270, 272, 312, 315, 356, and 358, and thus slight differences between the presented sequence and sequences in the prior art may exist and would be understood by one skilled in the art based on the present teachings.

An anti-CD20 antibody that is useful in the presently described methods is an anti-CD20 antibody, or antigen-binding fragment thereof including a heavy chain variable region (HCVR), light chain variable region (LCVR), and/or complementarity determining regions (CDRs), described in U.S. Pat. No. 7,608,693, which is incorporated by reference in its entirety.

In some instances, an anti-CD20 antibody or antigen-binding fragment useful in the presently described methods includes three HCDRs (HCDR1, HCDR2 and HCDR3) and three LCDRs (LCDR1, LCDR2 and LCDR3), wherein the HCDR1 includes the amino acid sequence of SEQ ID NO:1 (SYNMH); the HCDR2 includes the amino acid sequence of SEQ ID NO:2 (AIYPGNGDTSYNQKF); the HCDR3 includes the amino acid sequence of SEQ ID NO:3 (VVYYSNSYWYFDV); the LCDR1 includes the amino acid sequence of SEQ ID NO:4 (RASSSVSYMH); the LCDR2 includes the amino acid sequence of SEQ ID NO:5 (APSNLAS); and the LCDR3 includes the amino acid sequence of SEQ ID NO:6 (QQWSFNPPT).

In some instances, an anti-CD20 antibody or antigen-binding fragment useful in the presently described methods includes three HCDRs (HCDR1, HCDR2 and HCDR3) and three LCDRs (LCDR1, LCDR2 and LCDR3), wherein the HCDR1 includes the amino acid sequence of SEQ ID NO:19 (GYTFTSYNMH); the HCDR2 includes the amino acid sequence of SEQ ID NO:20 (AIYPGNGDTS); the HCDR3 includes the amino acid sequence of SEQ ID NO:3 (VVYYSNSYWYFDV); the LCDR1 includes the amino acid sequence of SEQ ID NO:4 (RASSSVSYMH); the LCDR2 includes the amino acid sequence of SEQ ID NO:5 (APSNLAS); and the LCDR3 includes the amino acid sequence of SEQ ID NO:6 (QQWSFNPPT).

In some instances, an anti-CD20 antibody or antigen-binding fragment useful in the presently described methods includes three HCDRs (HCDR1, HCDR2 and HCDR3) and three LCDRs (LCDR1, LCDR2 and LCDR3), wherein the HCDR1 includes the amino acid sequence of SEQ ID NO:21 (GYTFTSY); the HCDR2 includes the amino acid sequence of SEQ ID NO:22 (YPGNGD); the HCDR3 includes the amino acid sequence of SEQ ID N:3 (VVYYSNSYWYFDV); the LCDR1 includes the amino acid sequence of SEQ ID NO:4 (RASSSVSYMH); the LCDR2 includes the amino acid sequence of SEQ ID NO:5 (APSNLAS); and the LCDR3 includes the amino acid sequence of SEQ ID NO:6 (QQWSFNPPT).

TABLE 2
Amino Acid Sequences of CD20 CDRs (Kabat).

| SEQ | | | |
|---|---|---|---|
| ID | | | Positions |
| NO: | CDR | Sequence | (Kabat) |
| 1 | HC CDR1 Kabat | SYNMH | 26-35 |
| 2 | HC CDR2 Kabat | AIYPGNGDTSYNQKF | 50-64 |
| 3 | HC CDR3 Kabat | VVYYSNSYWYFDV | 99-111 |
| 4 | LC CDR1 Kabat | RASSSVSYMH | 24-33 |
| 5 | LC CDR2 Kabat | APSNLAS | 49-55 |
| 6 | LC CDR3 Kabat | QQWSFNPPT | 88-96 |

TABLE 3
Amino Acid Sequences of CD20 CDRs (AbM).

| SEQ | | | |
|---|---|---|---|
| ID | | | Positions |
| NO: | CDR | Sequence | (AbM) |
| 19 | HC CDR1 AbM | GYTFTSYNMH | 26-35 |
| 20 | HC CDR2 AbM | AIYPGNGDTS | 50-59 |
| 3 | HC CDR3 AbM | VVYYSNSYWYFDV | 99-111 |
| 4 | LC CDR1 AbM | RASSSVSYMH | 24-33 |
| 5 | LC CDR2 AbM | APSNLAS | 49-55 |
| 6 | LC CDR3 AbM | QQWSFNPPT | 88-96 |

TABLE 4
Amino Acid Sequences of CD20 CDRs (Chothia).

| SEQ | | | |
|---|---|---|---|
| ID | | | Positions |
| NO: | CDR | Sequence | (Chothia) |
| 21 | HC CDR1 | GYTFTSY | 26-32 |
| | Chothia | | |
| 22 | HC CDR2 | YPGNGD | 52-57 |
| | Chothia | | |
| 3 | HC CDR3 | VVYYSNS | 99-111 |
| | Chothia | YWYFDV | |
| 4 | LC CDR1 | RASSSVS | 24-33 |
| | Chothia | YMH | |
| 5 | LC CDR2 | APSNLAS | 49-55 |
| | Chothia | | |
| 6 | LC CDR3 | QQWSFNP | 88-96 |
| | Chothia | PT | |

In some instances, an anti-CD20 antibody or antigen-binding fragment useful in the presently described methods includes three HCDRs (HCDR1, HCDR2 and HCDR3) and three LCDRs (LCDR1, LCDR2 and LCDR3), wherein the amino acid sequences are encoded by the nucleotide sequences disclosed in U.S. Pat. No. 7,799,900 B2, which is incorporated by reference in its entirety.

In some instances, an anti-CD20 antibody or antigen-binding fragment useful in the presently described methods includes the heavy chain complementarity determining regions (HCDRs) of a heavy chain variable region (HCVR) of SEQ ID NOs: 7-13 and the light chain complementarity determining regions (LCDRs) of a light chain variable region (LCVR) of SEQ ID NOs: 14-17. SEQ ID NOs: 7-17 are shown in Table 5.

TABLE 5
Amino Acid Sequences of CD20 Heavy and Light Chains.

| SEQ | | |
|---|---|---|
| ID | | |
| NO: | Chain | Sequence |
| 7 | Heavy Chain | QAYLQQSGAELVRPGASVKMSCKASGYTFTSYN |
| | | MHWVKQTPRQGLEWIGAIYPGNGDTSYNQKFKG |
| | | KATLTVDKSSSTAYMQLSSLTSEDSAVYFCARVV |
| | | YYSNSYWYFDVWGTGTTVTVSS |
| 8 | Heavy Chain | EVQLVESGGGLVQPGGSLRLSCAASGYTFTSYNM |
| | | HWVRQAPGKGLEWVGAIYPGNGDTSYNQKFKGR |
| | | FTISVDKSKNTLYLQMNSLRAEDTAVYYCARVVY |
| | | YSNSYWYFDVWGQGTLVTVSS |
| 9 | Heavy Chain | EVQLVESGGGLVQPGGSLRLSCAASGYTFTSYNM |
| | | HWVRQAPGKGLEWVGAIYPGNGDTSYNQKFKGR |
| | | FTISVDKSKNTLYLQMNSLRAEDTAVYYCARVVY |
| | | YSASYWYFDVWGQGTLVTVSSASTKGPSVFPLAP |
| | | SSKSTSGGTAALGCLVKDYFPEPVTVSWNSGALT |
| | | SGVHTFPAVLQSSGLYSLSSVVTVPSSSLGTQTYIC |
| | | NVNHKPSNTKVDKKVEPKSCDKTHTCPPCPAPEL |
| | | LGGPSVFLFPPKPKDTLMISRTPEVTCVVVDVSHE |
| | | DPEVKFNWYVDGVEVHNAKTKPREEQYNATYRV |
| | | VSVLTVLHQDWLNGKEYKCKVSNKALPAPIAATI |
| | | SKAKGQPREPQVYTLPPSREEMTKNQVSLTCLVK |
| | | GFYPSDIAVEWESNGQPENNYKTTPPVLDSDGSFF |
| | | LYSKLTVDKSRWQQGNVFSCSVMHEALHNHYTQ |
| | | KSLSLSPGK |
| 10 | Heavy Chain | EVQLVESGGGLVQPGGSLRLSCAASGYTFTSYNM |
| | | HWVRQAPGKGLEWVGAIYPGNGATSYNQKFKGR |
| | | FTISVDKSKNTLYLQMNSLRAEDTAVYYCARVVY |
| | | YSASYWYFDVWGQGTLVTVSSASTKGPSVFPLAP |
| | | SSKSTSGGTAALGCLVKDYFPEPVTVSWNSGALT |
| | | SGVHTFPAVLQSSGLYSLSSVVTVPSSSLGTQTYIC |
| | | NVNHKPSNTKVDKKVEPKSCDKTHTCPPCPAPEL |
| | | LGGPSVFLFPPKPKDTLMISRTPEVTCVVVDVSHE |
| | | DPEVKFNWYVDGVEVHNAKTKPREEQYNATYRV |
| | | VSVLTVLHQDWLNGKEYKCKVSNKALPAPIAATI |
| | | SKAKGQPREPQVYTLPPSREEMTKNQVSLTCLVK |
| | | GFYPSDIAVEWESNGQPENNYKTTPPVLDSDGSFF |
| | | LYSKLTVDKSRWQQGNVFSCSVMHEALHNHYTQ |
| | | KSLSLSPGK |
| 11 | Heavy Chain | EVQLVESGGGLVQPGGSLRLSCAASGYTFTSYNM |
| | | HWVRQAPGKGLEWVGAIYPGNGATSYNQKFKGR |
| | | FTISVDKSKNTLYLQMNSLRAEDTAVYYCARVVY |
| | | YSASYWYFDVWGQGTLVTVSSASTKGPSVFPLAP |
| | | SSKSTSGGTAALGCLVKDYFPEPVTVSWNSGALT |
| | | SGVHTFPAVLQSSGLYSLSSVVTVPSSSLGTQTYIC |
| | | NVNHKPSNTKVDKKVEPKSCDKTHTCPPCPAPEL |
| | | LGGPSVFLFPPKPKDTLMISRTPEVTCVVVDVSHE |
| | | DPEVKFNWYVDGVEVHNAKTKPREEQYNATYRV |
| | | VSVLTVLHQDWLNGKEYKCKVSNKALPAPIAATI |
| | | SKAKGQPREPQVYTLPPSRDELTKNQVSLTCLVK |
| | | GFYPSDIAVEWESNGQPENNYKTTPPVLDSDGSFF |
| | | LYSKLTVDKSRWQQGNVFSCSVMHEALHNHYTQ |
| | | KSLSLSPGK |
| 12 | Heavy Chain | EVQLVESGGGLVQPGGSLRLSCAASGYTFTSYNM |
| | | HWVRQAPGKGLEWVGAIYPGNGATSYNQKFKGR |
| | | FTISVDKSKNTLYLQMNSLRAEDTAVYYCARVVY |
| | | YSASYWYFDVWGQGTLVTVSSASTKGPSVFPLAP |
| | | SSKSTSGGTAALGCLVKDYFPEPVTVSWNSGALT |
| | | SGVHTFPAVLQSSGLYSLSSVVTVPSSSLGTQTYIC |
| | | NVNHKPSNTKVDKKVEPKSCDKTHTCPPCPAPEL |
| | | LGGPSVFLFPPKPKDTLMISRTPEVTCVVVDVSHE |
| | | DPEVKFNWYVDGVEVHNAKTKPREEQYNATYRV |
| | | VSVLTVLHQDWLNGKEYKCAVSNKALPAPIEATI |
| | | SKAKGQPREPQVYTLPPSREEMTKNQVSLTCLVK |
| | | GFYPSDIAVEWESNGQPENNYKTTPPVLDSDGSFF |
| | | LYSKLTVDKSRWQQGNVFSCSVMHEALHNHYTQ |
| | | KSLSLSPGK |
| 13 | Heavy Chain | EVQLVESGGGLVQPGGSLRLSCAASGYTFTSYNM |
| | | HWVRQAPGKGLEWVGAIYPGNGATSYNQKFKGR |
| | | FTISVDKSKNTLYLQMNSLRAEDTAVYYCARVVY |
| | | YSASYWYFDVWGQGTLVTVSSASTKGPSVFPLAP |
| | | SSKSTSGGTAALGCLVKDYFPEPVTVSWNSGALT |
| | | SGVHTFPAVLQSSGLYSLSSVVTVPSSSLGTQTY |
| | | ICNVNHKPSNTKVDKKVEPKSCDKTHTCPPCPAP |
| | | ELLGGPSVFLFPPKPKDTLMISRTPEVTCVVVDV |
| | | SHEDPEVKFNWYVDGVEVHNAKTKPREEQYNATY |
| | | RVVSVLTVLHQDWLNGKEYKCKVSNAALPAPIAA |
| | | TISKAKGQPREPQVYTLPPSREEMTKNQVSLTCL |
| | | VKGFYPSDIAVEWESNGQPENNYKTTPPVLDSDG |
| | | SFFLYSKLTVDKSRWQQGNVFSCSVMHEALHNHY |
| | | TQKSLSLSPGK |
| 14 | Light Chain | QIVLSQSPAILSASPGEKVTMTCRASSSVSYMHWY |
| | | QQKPGSSPKPWIYAPSNLASGVPARFSGSGSGTSY |
| | | SLTISRVEAEDAATYYCQQWSFNPPTFGAGTKLEL |
| | | KR |
| 15 | Light Chain | DIQMTQSPSSLSASVGDRVTITCRASSSVSYMHWY |
| | | QQKPGKAPKPLIYAPSNLASGVPSRFSGSGSGTDF |
| | | TLTISSLQPEDFATYYCQQWSFNPPTFGQGTKVEI |
| | | KR |
| 16 | Light Chain | DIQMTQSPSSLSASVGDRVTITCRASSSVSYLHWY |
| | | QQKPGKAPKPLIYAPSNLASGVPSRFSGSGSGTDF |
| | | TLTISSLQPEDFATYYCQQWSFNPPTFGQGTKVEI |
| | | KRTVAAPSVFIFPPSDEQLKSGTASVVCLLNNFYP |
| | | REAKVQWKVDNALQSGNSQESVTEQDSKDSTYSLS |
| | | STLTLSKADYEKHKVYACEVTHQGLSSPVTKSFNR |
| | | GEC |
| 17 | Light Chain | DIQMTQSPSSLSASVGDRVTITCRASSSVSYLHWY |
| | | QQKPGKAPKPLIYAPSNLASGVPSRFSGSGSGTDF |
| | | TLTISSLQPEDFATYYCQQWAFNPPTFGQGTKVEI |
| | | KRTVAAPSVFIFPPSDEQLKSGTASVVCLLNNFYP |
| | | REAKVQWKVDNALQSGNSQESVTEQDSKDSTYSLS |
| | | STLTLSKADYEKHKVYACEVTHQGLSSPVTKSFNRG |
| | | EC |

In some instances, the anti-CD20 antibody or antigen-binding fragment thereof comprises any one of the heavy chain sequences selected from SEQ ID NOs: 7-13 and any one of the light chain sequences selected from SEQ ID NOs: 14-17. That is, the anti-CD20 antibody or antigen-binding fragment thereof can include any permutation of the combination of the heavy and light chains in Table 5 (e.g., SEQ ID NO:7 and SEQ ID NO:14; SEQ ID NO:7 and SEQ ID NO:15, and so on, until SEQ ID NO:13 and SEQ ID NO:17).

In some instances, an anti-CD20 antibody or antigen-binding fragment useful in the presently described methods includes the heavy chain complementarity determining regions (HCDRs) of a heavy chain variable region (HCVR) encoded by the nucleotide sequences disclosed in U.S. Pat. No. 7,799,900 B2, which is incorporated by reference in its entirety.

One useful anti-CD20 antibody or antigen-binding fragment thereof is, for example, one that specifically binds human CD20, including the amino acid sequence of SEQ ID NO:18. SEQ ID NO:18 is shown below:

(SEQ ID NO: 18)
MTTPRNSVNGTFPAEPMKGPIAMQSGPKPLFRRMSSLVGPTQSFF
MRESKTLGAVQIMNGLFHIALGGLLMIPAGIYAPICVTVWYPLWG
GIMYIISGSLLAATEKNSRKCLVKGKMIMNSLSLFAAISGMILSI
MDILNIKISHFLKMESLNFIRAHTPYINIYNCEPANPSEKNSPST
QYCYSIQSLFLGILSVMLIFAFFQELVIAGIVENEWKRTCSRPKS
NIVLLSAEEKKEQTIEIKEEVVGLTETSSQPKNEEDIEIIPIQEE
EEEETETNFPEPPQDQESSPIENDSSP

In some instances the anti-CD20 antibody or antigen-binding fragment thereof can in some instances specifically bind hCD20 with a KD of about 300 pM or less, as measured by surface plasmon resonance in a monomeric or dimeric assay. The antibody or antigen-binding portion thereof can in some instances exhibit a KD of about 200 pM or less, about 150 or less, about 100 pM or less, or about 50 pM. In some instances, the antibody or antigen-binding fragment blocks CD20 activity with an IC50 of about 100 pM or less, as measured by luciferase bioassay. In some instances, the antibody or antigen-binding fragment exhibits an IC50 of about 50 pM or less, about 30 pM or less, or about 25 pM or less, as measured by STAT6 luciferase bioassay. The antibody or antigen-binding fragment can, in some instances, block hCD20 activity with an IC50 of about 100 pM or less, about 90 pM or less, about 50 pM or less, or about 20 pM or less, as measured by STAT6 luciferase bioassay.

Of particular, use in the presently described methods is the anti-CD20 antibody referred to and known in the art as Ocrevus®, or a bioequivalent thereof.

Other anti-CD20 antibodies that can be used in the context of the methods of the present disclosure include, e.g., the antibody referred to and known in the art as Rituxan (rituximab), Kesimpta (ofatumumab), Gazyva (obinutuzumab), Zevalin (ibritumomab), Y-90 Zevalin (ibritumomab), Truxima, Ruxience, Rituxan Hycela (hyaluronidase/rituximab), Riabni, In-111 Zevalin (ibritumomab), Briumvi (ublituximab) Bexxar (iodine i 131 tositumomab), and Arzerra (ofatumumab).

Anti-CD20 antibodies useful in the presently-described methods can have pH-dependent binding characteristics. For example, an anti-CD20 antibody can exhibit reduced binding to CD20 at acidic pH as compared to neutral pH. Alternatively, an anti-CD20 antibody can exhibit enhanced binding to its antigen at acidic pH as compared to neutral pH. The expression “acidic pH” includes pH values less than about 6.2, e.g., about 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding to CD20 at acidic pH as compared to neutral pH” is expressed in terms of a ratio of the KD value of the antibody binding to CD20 at acidic pH to the KD value of the antibody binding to CD20 at neutral pH (or vice versa). For example, an antibody or antigen-binding fragment thereof can be regarded as exhibiting “reduced binding to CD20 at acidic pH as compared to neutral pH” for purposes of the present disclosure if the antibody or antigen-binding fragment thereof exhibits an acidic/neutral KD ratio of about 3.0 or greater. In certain instances, the acidic/neutral KD ratio for an antibody or antigen-binding fragment of the present disclosure can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0, 25.0, 30.0, 40.0, 50.0, 60.0, 70.0, 100.0, or greater.

Antibodies with pH-dependent binding characteristics can be obtained, e.g., by screening a population of antibodies for reduced (or enhanced) binding to a particular antigen at acidic pH as compared to neutral pH. Additionally, modifications of the antigen-binding domain at the amino acid level can yield antibodies with pH-dependent characteristics. For example, by substituting one or more amino acids of an antigen-binding domain (e.g., within a CDR) with a histidine residue, an antibody with reduced antigen-binding at acidic pH relative to neutral pH can be obtained. As used herein, the expression “acidic pH” means a pH of 6.0 or less.

### II. Pharmaceutical Compositions

The present disclosure provides methods that include administering an anti-CD20 antibody to a subject, wherein the antibody is an ingredient in a pharmaceutical composition. Useful pharmaceutical compositions can be formulated with suitable carriers, excipients, and other agents that provide suitable transfer, delivery, tolerance, and the like. A multitude of appropriate formulations can be found in the formulary Remington's Pharmaceutical Sciences, Mack Publishing Company, Easton, Pa. See also Powell et al. “Compendium of excipients for parenteral formulations” PDA (1998) J Pharm Sci Technol 52:238-311. Various delivery systems are known and can be used to administer a pharmaceutical composition, e.g., encapsulation in liposomes, microparticles, and microcapsules (see, e.g., Wu et al., 1987, J. Biol. Chem. 262:4429-4432).

Methods of administration include, but are not limited to, intradermal, intramuscular, intraperitoneal, intravenous, subcutaneous, intranasal, epidural, and oral routes. In some instances, administration is subcutaneous. The composition can be administered by any convenient route, for example by infusion or bolus injection, by absorption through epithelial or mucocutaneous linings (e.g., oral mucosa, rectal and intestinal mucosa, etc.) and can be administered together with other biologically active agents.

In some instances, the compositions disclosed herein (e.g., having CD20 binding antibodies) are administered to a human patient in accord with known methods, such as by intravenous administration, e.g., as a bolus or by continuous infusion over a period of time, by subcutaneous, intramuscular, intraperitoneal, intracerobrospinal, intra-articular, intrasynovial, intrathecal, or inhalation routes, generally by intravenous or subcutaneous administration.

A pharmaceutical composition can be delivered subcutaneously or intravenously with a standard needle and syringe. In some embodiments, the syringe is a prefilled syringe. With respect to subcutaneous delivery, a syringe (e.g., a prefilled syringe) or a pen delivery device, such as a prefilled pen or an autoinjector, has applications in delivering a pharmaceutical composition in the presently described methods. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded. In some instances, the prefilled pen delivery device is an autoinjector.

In certain situations, the pharmaceutical composition can be delivered in a controlled release system. In some instances, a pump can be used. In some instances, polymeric materials can be used; see, Medical Applications of Controlled Release, Langer and Wise (eds.), 1974, CRC Pres., Boca Raton, Fla. In some instances, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

Injectable preparations are of particular use in the presently described methods. Injectable preparations can include dosage forms for intravenous, subcutaneous, intracutaneous and intramuscular injections, drip infusions, etc. These injectable preparations can be prepared using known methods. For example, injectable preparations can be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which can be used in combination with an appropriate solubilizing agent such as an alcohol (e.g., ethanol), a polyalcohol (e.g., propylene glycol, polyethylene glycol), a nonionic surfactant (e.g., polysorbate 80, HCO-50 (polyoxyethylene (50 mol) adduct of hydrogenated castor oil)}, etc. Useful oily media include, e.g., sesame oil and/or soybean oil, which can be used in combination with a solubilizing agent such as benzyl benzoate and/or benzyl alcohol. In some instances, the pharmaceutical composition is disposed in an appropriate ampoule.

Pharmaceutical compositions for oral or parenteral use described above can be prepared into dosage forms in a unit dose suited to fit a dose of the active ingredients. Such dosage forms in a unit dose include, for example, tablets, pills, capsules, injections (ampoules), suppositories, etc.

Exemplary pharmaceutical compositions including an anti-CD20 antibody that can be used in the context of the present disclosure are disclosed, e.g., in U.S. Pat. No. 7,799,900 B2, the disclosure of which is incorporated herein by reference in its entirety.

### III. Methods of Generating Anti-CD20 Antibodies and Fragments

Monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium which medium preferably contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which substances prevent the growth of HGPRT-deficient cells.

Preferred fusion partner myeloma cells are those that fuse efficiently, support stable high-level production of antibody by the selected antibody-producing cells, and are sensitive to a selective medium that selects against the unfused parental cells. Preferred myeloma cell lines are murine myeloma lines, such as those derived from MOPC-21 and MPC-11 mouse tumors available from the Salk Institute Cell Distribution Center, San Diego, Calif. USA, and SP-2 and derivatives e.g., X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); and Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The binding affinity of the monoclonal antibody can, for example, be determined by the Scatchard analysis described in Munson et al., Anal. Biochem., 107:220 (1980).

Once hybridoma cells that produce antibodies of the desired specificity, affinity, and/or activity are identified, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal e.g, by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose®) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Plückthun, Immunol. Revs., 130:151-188 (1992).

In a further embodiment, monoclonal antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA that encodes the antibody may be modified to produce chimeric or fusion antibody polypeptides, for example, by substituting human heavy chain and light chain constant domain (CH and CL) sequences for the homologous murine sequences (U.S. Pat. No. 4,816,567; and Morrison, et al., Proc. Natl. Acad. Sci. USA, 81:6851 (1984)), or by fusing the immunoglobulin coding sequence with all or part of the coding sequence for a non-immunoglobulin polypeptide (heterologous polypeptide). The non-immunoglobulin polypeptide sequences can substitute for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

Additional methods of productions are described in U.S. Pat. No. 7,799,900 B2, which is incorporated by reference in its entirety.

Methods of Identifying Disorders Treatable with Anti-CD20 Antibodies

As discussed above, presently-described new indications that are treatable with an anti-CD20 antibody were, at least in part, identified using computational methods. In some instances, the methods disclosed herein include identifying a disorder that is treatable with an anti-CD20 antibody. In some instances, the methods of identifying a disorder include (a) selecting a characteristic or a plurality of characteristics related to a CD20 pathway in a set of data representing medical records of a plurality of subjects; (b) clustering, by a computer system, in accordance with the characteristic or the plurality of characteristics related to the CD20 pathway, a subset of subjects from the plurality of subjects, wherein the subset comprises the characteristic or the plurality of characteristics; (c) identifying in the subset clustered in step (b) the CD20-related disorder based on symptoms associated with the characteristic or the plurality of characteristics related to the CD20 pathway; and (d) selecting the subject having at least one symptom of, or determined to be susceptible to, a CD20-related disorder identified in step (c). In some instances, the method further comprises administering a therapeutically effective amount of the anti-CD20 antibody to the subject, wherein the anti-CD20 antibody comprises: a variable heavy chain CDR1 of SEQ ID NO:1 (SYNMH); a variable heavy chain CDR2 of SEQ ID NO:2 (AIYPGNGDTSYNQKF); a variable heavy chain CDR3 of SEQ ID NO:3 (VVYYSNSYWYFDV); a variable light chain CDR1 of SEQ ID NO:4 (RASSSVSYMH); a variable light chain CDR2 of SEQ ID NO:5 (APSNLAS); and a variable light chain CDR3 of SEQ ID NO:6 (QQWSFNPPT).

A general description of the methods used for identifying indications described herein is provided below.

### I. Background

The computation methods described herein can be used to find new clinical indications (e.g., a reason to use a drug) for clinically approved drugs. Guided by relevant clinical questions, powerful advanced analytics techniques can mine clinically relevant information hidden in large amounts of data, which can then assist clinical decision making.

The computational methods described herein can use similarity measures (chemical similarity, molecular activity similarity, gene expression similarity, or side effect similarity), molecular docking, or shared molecular pathology to detect new drug-disease relationships. The computational approaches can be classified as network-based, text mining (literature search), and semantic approaches.

Data processing systems and methods generally described in this specification can be used to identify potential indications that have not been previously identified using conventional techniques.

In the following description, for the purposes of explanation, numerous specific details are set forth in order to provide a thorough understanding of the present disclosure. It will be apparent, however, that the present disclosure may be practiced without these specific details. In other instances, well-known structures and devices are shown in block diagram form in order to avoid unnecessarily obscuring the present disclosure.

In the drawings, specific arrangements or orderings of schematic elements, such as those representing devices, modules, instruction blocks and data elements, are shown for ease of description. However, it should be understood by those skilled in the art that the specific ordering or arrangement of the schematic elements in the drawings is not meant to imply that a particular order or sequence of processing, or separation of processes, is required. Further, the inclusion of a schematic element in a drawing is not meant to imply that such element is required in all implementations or that the features represented by such element may not be included in or combined with other elements in some implementations.

Further, in the drawings, where connecting elements, such as solid or dashed lines or arrows, are used to illustrate a connection, relationship, or association between or among two or more other schematic elements, the absence of any such connecting elements is not meant to imply that no connection, relationship, or association can exist. In other words, some connections, relationships, or associations between elements are not shown in the drawings so as not to obscure the disclosure. In addition, for ease of illustration, a single connecting element is used to represent multiple connections, relationships or associations between elements. For example, where a connecting element represents a communication of signals, data, or instructions, it should be understood by those skilled in the art that such element represents one or multiple signal paths (e.g., a bus), as may be needed, to affect the communication.

Reference will now be made in detail to implementations, examples of which are illustrated in the accompanying drawings. In the following detailed description, numerous specific details are set forth in order to provide a thorough understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

Several features are described hereafter that can each be used independently of one another or with any combination of other features. However, any individual feature may not address any of the problems discussed above or might only address one of the problems discussed above. Some of the problems discussed above might not be fully addressed by any of the features described in this specification. Although headings are provided, data related to a particular heading, but not found in the section having that heading, may also be found elsewhere in this description.

### II. Example Data Processing Systems and Methods

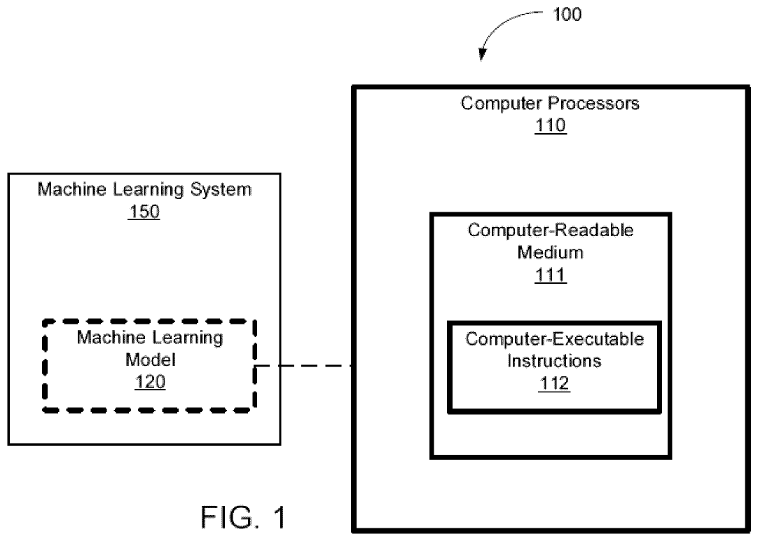

FIG. 1 shows an example of a data processing system 100. In some implementations, the data processing system 100 is configured to process data that can represent medical records of a plurality of patients to identify new indications of a drug (a drug for treating novel indications or for repurposing). The system 100 includes computer processors 110. The computer processors 110 include computer-readable memory 111 and computer readable instructions 112. The system 100 also includes a machine learning system 150. The machine learning system 150 includes a machine learning model 120. The machine learning model 120 can be separate from or integrated with the computer processors 110.

The computer-readable medium 111 (or computer-readable memory) can include any data storage technology type which is suitable to the local technical environment, including but not limited to semiconductor based memory devices, magnetic memory devices and systems, optical memory devices and systems, fixed memory, removable memory, disc memory, flash memory, dynamic random-access memory (DRAM), static random-access memory (SRAM), electronically erasable programmable read-only memory (EEPROM) and the like. In some implementations, the computer-readable medium 111 includes code-segment having executable instructions.

In some implementations, the computer processors 110 include a general purpose processor. In some implementations, the computer processors 110 include a central processing unit (CPU). In some implementations, the computer processors 110 include at least one application specific integrated circuit (ASIC). The computer processors 110 can also include general purpose programmable microprocessors, graphic processing units, special-purpose programmable microprocessors, digital signal processors (DSPs), programmable logic arrays (PLAs), field programmable gate arrays (FPGA), special purpose electronic circuits, etc., or a combination thereof. The computer processors 110 are configured to execute program code such as the computer-executable instructions 112 and configured to execute executable logic that includes the machine learning model 120.

The computer processors 110 are configured to receive data representing medical records of a plurality of patients. For example, the computer processors 110 can receive data from a database that includes electronic medical records (EMRs) data for approximately 94 million patients (or more) identifiable by a key identifier (ID) that allows matching of patients across different data tables. In some implementations, the data indicates diagnosis, lab test, procedures, medications, patient events, insurance, biomarkers, measurements, clinical status, lifestyle parameters, microbiology, prescriptions, and so forth. In some implementations, the data includes natural language process driven data. The data can be received through any of various techniques, such as wireless communications, optical fiber communications, USB, CD-ROM, and so forth.

The machine learning system 150 is capable of applying machine learning techniques to train the machine learning model 120. As part of the training of the machine learning model 120, the machine learning system 150 can form a training set of input data by identifying a positive training set of input data items that have been determined to have the property in question, and, in some implementations, can form a negative training set of input data items that lack the property in question.

The machine learning system 150 extracts feature values from the input data of the training set, the features being variables deemed potentially relevant to whether or not the input data items have the associated property or properties. An ordered list of the features for the input data is herein referred to as the feature vector for the input data. In some implementations, the machine learning system 150 applies dimensionality reduction to reduce the amount of data in the feature vectors for the input data to a smaller, more representative set of data. For example, the machine learning system 150 can apply Multiple Correspondence Analysis (MCA), linear discriminant analysis (LDA), principal component analysis (PCA), and so forth.

In some implementations, the machine learning system 150 uses unsupervised machine learning to train the machine learning model 120. Typically, unsupervised machine learning techniques make inferences from datasets using input vectors without referring to known, or labelled, outcomes. In some implementations, the machine learning system 150 can perform clustering to divide data points into a number of groups such that the data points in the same group are more similar to other data points in the same group and dissimilar to data points in other groups. In some implementations, clustering includes performing K-means clustering, in which a one-level unnested partitioning of data points is created by iteratively partitioning the data set. That is, if K is the desired number of clusters, in each iteration, the data set is partitioned into K disjoint clusters. The processes can be continued until a specified clustering criterion function value is optimized. In some implementations, the machine learning system 150 is configured to perform bisecting K-means clustering. Bisecting k-means clustering typically involves splitting one cluster into two subclusters at each bisecting step (e.g., by using k-means) until k clusters are obtained. Bisecting K-means clustering may be more beneficial when compared to K-means clustering, as bisecting K-means clustering can reduce computation time when K is a relatively large value, can produce clusters of similar size, and can produce clusters with smaller entropy.

The computer processors 110 are configured to execute the computer-executable instructions 112 to perform one or more operations. In some implementations, the one or more operations include receiving data representing medical records of a plurality of patients. For example, the computer processors 110 can receive data from a database that includes electronic medical records (EMRs) for approximately 94 million patients (or more) identifiable by a key identifier (ID) that allows matching of patients across different data tables. In some implementations, the data indicates diagnosis, lab test, procedures, medications, patient events, insurance, biomarkers, measurements, clinical status, lifestyle parameters, microbiology, prescriptions, and so forth. In some implementations, the data includes natural language process driven data. The data can be received through any of various techniques, such as wireless communications, optical fiber communications, USB, CD-ROM, and so forth.

In some implementations, the one or more operations include selecting, based on the medical records, a set of patients. Selection of the set of patients includes determining at least one target signaling pathway associated with a drug. For example, if the drug is Ocrevus®, the computer processors 110 can determine that the drug modulates the CD20 signaling pathway based on known functions of the drug. Selecting the set of patients also includes determining one or more indicators based on one or more factors corresponding to a diagnosis linked to the target signaling pathway. For example, factors such as pathway mechanisms, related clinical conditions, therapeutic analogues, data and epidemiology, and pharmaceutical life-cycle management alignment can be used to search through sources that include medical databases and medical evidence software to identify diseases linked to the determined signaling pathway. These diseases can be categorized based on the strength of the link to the determined signaling pathway. The categories can include a focused group, a medium group, and a broad group. For example, returning to CD20 example, a focused group of diseases can include diseases that have a direct relationship with the CD20 mechanism of action in B-cells, a medium group of diseases can include diseases that have an indirect relationship with the CD20 mechanism of action other non-B-cells, and a broad group of diseases can include diseases associated with a broader inflammatory response. Moving from the focus group to the broad group can increase the number of indicators to be considered when selecting the set of patients, and can reduce the likelihood of molecule impact. Accordingly, in some implementations, only the focused group, or the focused and medium group, are used to select the set of patients. In some implementations, only patients with at least one diagnosis, medication, lab test, and/or procedure associated with the determined signaling pathway are selected for inclusion into the set of patients. A detailed example of factors and indicators is provided later in the examples.

In some implementations, the one or more operations include determining a plurality of patient characteristics (sometimes referred to as features in this specification) of the set of patients, in which each patient of the set of patients exhibits at least one of the plurality of patient characteristics. Determining the plurality of patient characteristics can include analyzing the initially received data to identify broad patient characteristics to capture all or a substantial portion of the received data. For example, the broad patient characteristics can correspond to diagnoses (e.g., immuno-conditions, diabetes), prescriptions (e.g., immuno-drugs, other drug classes), procedures (e.g., human leukocyte antigen typing), and laboratory results (e.g., IgE abnormal high/low). In some implementations, determining the plurality of patient characteristics includes receiving user input (e.g., through a user interface in communication with the computer processors 110). For example, a user can input patient characteristics based on clinical input, demographics, medication, comorbidities, procedures, and laboratory tests data specific to immunology. Bespoke characteristic classes may also be added to increase data completeness, representativeness, and to collect more information on diseases and drug response. In some implementations, determining the plurality of patient characteristics includes validating the plurality of patient characteristics. Validating can include determining whether the patient characteristics of the initially received data are mapped correctly to the selected set of patients by calculating the percentage of selected patients with at least one of each characteristic family (e.g., the percentage of patients with a prescription record) and comparing this percentage to the percentage of patients of the initially received data with at least one of each characteristic family. The two numbers being closer in value indicates that the mapping has been done correctly. Validating can include determining whether the patient characteristics have been mapped to the correct patient by identifying a number of patients that are included in both the initially received data and the selected set of patients to verify identical mapping of patient characteristics between the patients of the initially received data and the selected set of patients.

In some implementations, the one or more operations include grouping, in accordance with the plurality of patient characteristics (e.g., as defined by features related to the determined signaling pathway), the set of patients to generate a plurality of distinct groups in which each of the distinct groups include at least one patient of the set of patients. For example, the one or more computer processors 110 can execute the machine learning model 120 to perform a clustering technique, such as the bisecting k-means clustering technique described above. The clustering can result in a plurality of clusters (e.g., distinct groups) of patients in which patients in one cluster are more similar to each other than patients in other clusters with respect to their corresponding patient characteristics. In some implementations, the generated clusters may show correlations among patient characteristics, even if they weren't present in the same patient. Clinical inputs can be received and used in various stages of the clustering process to ensure the clinical relevance of the resulting clusters. For example, disease experts' clinical inputs can facilitate the creation of clinically relevant cohorts, in the inclusion and grouping of clinically relevant features, and in validating and assessing the clusters. Patient characteristics can be identified as being distinctive in clusters if they occurred more frequently than in the general population (e.g., overall in the selected set of patients).

In some implementations, Multiple Correspondence Analysis (MCA) is used to reduce the dimensions of the patient characteristics. Bisecting K-means can facilitate an appropriate and effective separation of patients with sufficiently “tight” but stable clusters, and allow a large number of clusters that exhibited immuno-relatedness to be used for scoring the patient characteristics, which is explained later in more detail. The resulting clusters can be presented (e.g., through a user interface) to users (e.g., clinical experts) for validation and assessment. This can reduce the risk of non-interpretability of the clusters and to ensure the absence of overlapping features between the different clusters.

In some implementations, the one or more operations include selecting, based on one or more group selection criteria, a set of distinct groups of the plurality of distinct groups. In some implementations, selecting the set of distinct groups includes ranking the groups and selecting a number of the most highly ranked groups (e.g., the top 60 ranked groups). The groups can be ranked based on immunology enrichment, stability, purity, and size. In some implementations, one or more measures (sometimes referred to as feature scores in this specification) are calculated for each patient characteristic to rank the clusters. The one or more measures can include, for example, distinctiveness (sometimes referred to as “lift score” in this specification), the number of patients within a cluster that present the patient characteristic, and an immunology score. The distinctiveness score measure how distinctive a patient characteristic is within a cluster versus the rest of the population (e.g., if males represent 50% of the population and 75% of the cluster, then the “lift score” can be equal to 1.5). In some implementations, only patient characteristics with a lift score that exceeds a threshold lift score (e.g., 1) and appearing in a percentage of patients that exceed a threshold percentage of patients (e.g., 10%) are considered to define clusters and correspond to a theme of the clusters. Patient characteristics that are considered to define a cluster may be referred to as potentially relevant patient characteristics in this specification. The patient characteristics (e.g., either the patient characteristics considered to define clusters or all of the patient characteristics) can then be given an immunology score, which scores the patient characteristics according to its type (e.g., disease, drug, laboratory test, procedure, and so forth) and immunology relevance. The patient characteristic scores within each cluster can then be aggregated (e.g., summed) and normalized. Clusters meeting a threshold cluster score (e.g., 50%) can then be considered as immunology-specific.

Selecting the set of distinct groups can include assessing one or more of the stability, purity, and the number of patients within each cluster. Stability can be assessed using one or more of the following methods: (1) reproducing the clusters on different sizes of data; (2) changing the initializing seeds of the clusters; (3) changing the number of clusters produced and (4) applying a training-test method. For each cluster in the training set, stability can be defined as the maximum proportion of patients that are also grouped together in the test set. Purity can be measured by the intra-cluster variance of MCA components of patients within a cluster, which can result in homogenous and dense clusters. In some implementations, a cluster is selected if it exceeds a threshold stability percentage (e.g., 50%) and exceeds a threshold purity percentage (e.g., the cluster is in the highest 20% of purity among all clusters).

In some implementations, the one or more operations include identifying one or more relevant patient characteristics (e.g., indications) by analyzing each distinct group of the set of distinct groups. Identifying one or more relevant patient characteristics can include ranking the patient characteristics presented by each selected cluster (e.g., all of the patient characteristics or the patient characteristics considered to define a cluster). The ranking can be based on the frequency of co-occurrence with each of a number of established (reference) characteristics (referential) of the drug (e.g., if the drug is Ocrevus®, the reference characteristics may include asthma, atopic dermatitis, IgE allergy, and a composite immunology score). The co-occurrence can be measured by calculating the proportion of patient-weighted clusters that contain both the patient characteristic and the referential. In some implementations, one or more patient characteristics judged by subject-matter experts as relevant to the core cluster theme (e.g., as indicated by user input received through a user interface) can also be considered for evaluation, regardless of the number of patients in which these features appeared (might be <10%).

Identifying one or more patient characteristics can include assessing clinical and commercial feasibility of the patient characteristics. For example, patient characteristics that show a distinct clinical diagnosis can be identified. Commercial assessment can be based on data indicating forecast sales and competitor assets were available, a determined link to the targeted signal pathway (whether found or not in publications), worldwide prevalence of the patient characteristic, and the disability-adjusted life year (DALY) of the patient characteristic (e.g., per 100,000 life years). As a result, in some implementations, the one or more operations generally output new indications for the drug.

While this specification here generally describes a patient as a human patient, implementations are not so limited. For example, a patient can refer to a non-human animal.

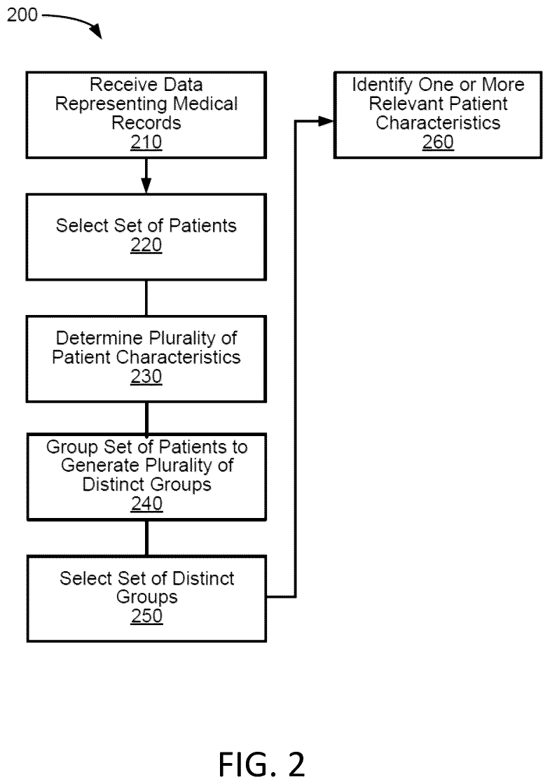

FIG. 2 is a flow chart illustrating an example method 200 for identifying indications for treatment with a drug. The method 200 can be performed by the data processing system 100 described previously with reference to FIG. 1. The method 200 includes receiving data representing medical records (block 210), selecting a set of patients (block 220), determining a plurality of patient characteristics (block 230), grouping the set of patients to generate a plurality of distinct groups (block 240), selecting a set of distinct groups (block 250), and identifying one or more relevant patient characteristics (block 260).

At block 210, data representing medical records of a plurality of patients is received. The data can be received from, for example, a database that includes EMRs for approximately 94 million patients (or more) identifiable by a key ID that allows matching of patients across different data tables. In some implementations, the data indicates diagnosis, lab test, procedures, medications, patient events, insurance, biomarkers, measurements, clinical status, lifestyle parameters, microbiology, prescriptions, and so forth. In some implementations, the data includes natural language process driven data. The data can be received through any of various techniques, such as wireless communications, optical fiber communications, USB, CD-ROM, and so forth.

At block 220, at least one target signaling pathway associated with the drug is determined. For example, if the drug is Ocrevus®, it can be determined that the drug modulates the CD20 signaling pathway based on known functions of the drug. In some implementations, one or more indicators are determined based on one or more factors corresponding to a diagnosis linked to the target signaling pathway. For example, factors such as pathway mechanisms, related clinical conditions, therapeutic analogues, data and epidemiology, and pharmaceutical life-cycle management alignment can be used to search through sources that include medical databases and medical evidence software to identify diseases linked to the determined signaling pathway. These diseases can be categorized based on the strength of the link to the determined signaling pathway. The categories can include a focused group, a medium group, and a broad group. For example, returning to the CD20 example, a focused group of diseases can include diseases that have a direct relationship with the CD20 mechanism of action in B cells, a medium lens group of diseases can include diseases that have indirect relationship with the CD20 mechanism of action in non-B cells, and a broad group of diseases can include diseases associated with a broader inflammatory response. Moving from the focus group to the broad group can increase the number of indicators to be considered when selecting the set of patients, and can reduce the likelihood of molecule impact. Accordingly, in some implementations, only the focused group, or the focused and medium group, are used to select the set of patients. In some implementations, only patients with at least one diagnosis, medication, lab test, and/or procedure associated with the determined signaling pathway are selected for inclusion into the set of patients.

At block 230, a plurality of patient characteristics of the set of patients is determined, in which each patient of the set of patients exhibits at least one of the plurality of patient characteristics. Determining the plurality of patient characteristics can include analyzing the initially received data to identify broad patient characteristics to capture all or a substantial portion of the received data. For example, the broad patient characteristics can correspond to diagnoses (e.g., immuno-conditions, diabetes), prescriptions (e.g., immuno-drugs, other drug classes), procedures (e.g., human leukocyte antigen typing), and laboratory results (e.g., IgE abnormal high/low). In some implementations, determining the plurality of patient characteristics includes receiving user input (e.g., through a user interface). For example, a user can input patient characteristics based on clinical input and demographics, medication, comorbidities, procedures and laboratory tests data specific to immunology. Bespoke characteristic classes may also be added to increase data completeness, representativeness, and to collect more information on diseases and drug response. In some implementations, determining the plurality of patient characteristics includes validating the plurality of patient characteristics. Validating can include determining whether the patient characteristics of the initially received data are mapped correctly to the selected set of patients by calculating the percentage of selected patients with at least one of each characteristic family (e.g., the percentage of patients with a prescription record) and comparing this percentage to the percentage of patients of the initially received data with at least one of each characteristic family. The two numbers being closer in value indicates that the mapping has been done correctly. Validating can include determining whether the patient characteristics have been mapped to the correct patient by identifying a number of patients that are included in both the initially received data and the selected set of patients to verify identical mapping of patient characteristics between the patients of the initially received data and the selected set of patients.

At block 240, the set of patients are grouped in accordance with the plurality of patient characteristics (e.g., as defined by features related to the determined signaling pathway) to generate a plurality of distinct groups in which each of the distinct groups include at least one patient of the set of patients. For example, clustering techniques, such as the bisecting k-means clustering technique described above, can be performed on the set of patients using the plurality of patient characteristics. The clustering can result in a plurality of clusters (e.g., distinct groups) of patients in which patients in one cluster are more similar to each other than patients in other clusters with respect to their corresponding patient characteristics. In some implementations, the generated clusters may show correlations among patient characteristics, even if they weren't present in the same patient. Clinical inputs can be received and used in various stages of the clustering process to ensure the clinical relevance of the resulting clusters. For example, disease experts' clinical inputs can facilitate the creation of clinically relevant cohorts, in the inclusion and grouping of clinically relevant features, and in validating and assessing the clusters. Patient characteristics can be identified as being distinctive in clusters if they occurred more frequently than in the general population (e.g., overall in the selected set of patients).

In some implementations, Multiple Correspondence Analysis (MCA) is used to reduce the dimensions of the patient characteristics. Bisecting K-means can facilitate an appropriate and effective separation of patients with sufficiently “tight” but stable clusters, and allow a large number of clusters that exhibited immuno-relatedness to be used for scoring the patient characteristics, which is explained later in more detail. The resulting clusters can be presented (e.g., through a user interface) to users (e.g., clinical experts) for validation and assessment. This can reduce the risk of non-interpretability of the clusters and to ensure the absence of overlapping features between the different clusters.

At block 250, a set of distinct groups of the plurality of distinct groups is selected based on or more group selection criteria. In some implementations, selecting the set of distinct groups includes ranking the groups and selecting a number of the most highly ranked groups (e.g., the top 60 ranked groups). The groups can be ranked based on immunology enrichment, stability, purity, and size. In some implementations, one or more measures are calculated for each patient characteristic to rank the clusters. The one or more measures can include, for example, distinctiveness (sometimes referred to as “lift score” in this specification), the number of patients within a cluster that present the patient characteristic, and an immunology score. The distinctiveness score measure how distinctive a patient characteristic is within a cluster versus the rest of the population (e.g., if males represent 50% of the population and 75% of the cluster, then the “lift score” can be equal to 1.5). In some implementations, only patient characteristics with a lift score that exceeds a threshold lift score (e.g., 1) and appearing in a percentage of patients that exceed a threshold percentage of patients (e.g., 10%) are considered to define clusters and correspond to a theme of the clusters. Patient characteristics that are considered to define a cluster may be referred to as potentially relevant patient characteristics in this specification. The patient characteristics (e.g., either the considered patient characteristics for defining clusters or all of the patient characteristics) can then be given an immunology score, which scores the patient characteristics according to its type (e.g., disease, drug, laboratory test, procedure, and so forth) and immunology relevance. The patient characteristic scores within each cluster can then be aggregated (e.g., summed) and normalized. Clusters meeting a threshold cluster score (e.g., 50%) can then be considered as immunology-specific.

Selecting the set of distinct groups can include assessing one or more of the stability, purity, and the number of patients within each cluster. Stability can be assessed using one or more of the following methods: (1) reproducing the clusters on different sizes of data; (2) changing the initializing seeds of the clusters; (3) changing the number of clusters produced and (4) applying a training-test method. For each cluster in the training set, stability can be defined as the maximum proportion of patients that are also grouped together in the test set. Purity can be measured by the intra-cluster variance of MCA components of patients within a cluster, which can result in homogenous and dense clusters. In some implementations, a cluster is selected if it exceeds a threshold stability percentage (e.g., 50%) and exceeds a threshold purity percentage (e.g., the cluster is in the highest 20% of purity among all clusters).

At block 260, one or more relevant patient characteristics are identified by analyzing each distinct group of the set of distinct groups. Identifying one or more relevant patient characteristics can include ranking the patient characteristics presented by each selected cluster (e.g., all of the patient characteristics or the patient characteristics considered to define a cluster). The ranking can be based on the frequency of co-occurrence with each of a number of established (reference) characteristics (referential) of the drug (e.g., if the drug is Ocrevus®, the reference characteristics may include asthma, atopic dermatitis, IgE allergy, and a composite immunology score). The co-occurrence can be measured by calculating the proportion of patient-weighted clusters that contain both the patient characteristic and the referential. In some implementations, one or more patient characteristics judged by subject-matter experts as relevant to the core cluster theme (e.g., as indicated by user input received through a user interface) can also be considered for evaluation, regardless of the number of patients in which these features appeared (might be <10%).

Identifying one or more patient characteristics can include assessing clinical and commercial feasibility of the patient characteristics. For example, patient characteristics that show a distinct clinical diagnosis can be identified. Commercial assessment can be based on data indicating forecast sales and competitor assets were available, a determined link to the targeted signal pathway (whether found or not in publications), worldwide prevalence of the patient characteristic, and the disability-adjusted life year (DALY) of the patient characteristic (e.g., per 100,000 life years).

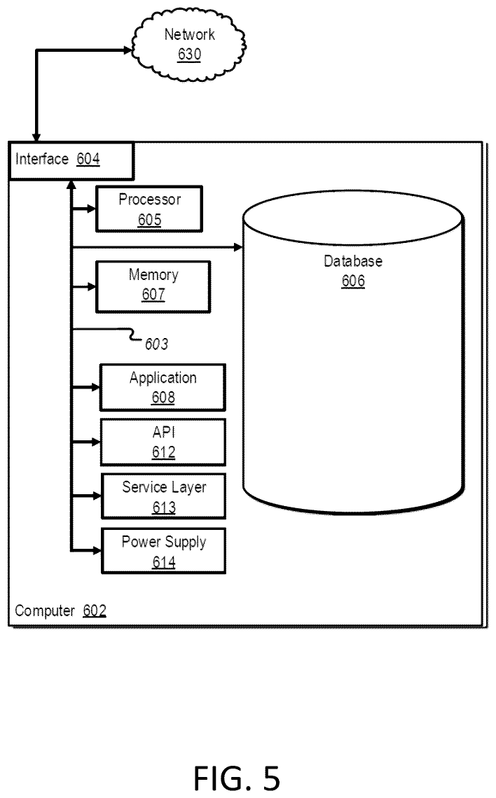

FIG. 5 is a block diagram of an example computer system 600 used to provide computational functionalities associated with described algorithms, methods, functions, processes, flows, and procedures described in the present disclosure (such as the method 200 described previously with reference to FIG. 2), according to some implementations of the present disclosure. The illustrated computer 602 is intended to encompass any computing device such as a server, a desktop computer, a laptop/notebook computer, a wireless data port, a smart phone, a personal data assistant (PDA), a tablet computing device, or one or more processors within these devices, including physical instances, virtual instances, or both. The computer 602 can include input devices such as keypads, keyboards, and touch screens that can accept user information. Also, the computer 602 can include output devices that can convey information associated with the operation of the computer 602. The information can include digital data, visual data, audio information, or a combination of information. The information can be presented in a graphical user interface (UI) (or GUI).

The computer 602 can serve in a role as a client, a network component, a server, a database, a persistency, or components of a computer system for performing the subject matter described in the present disclosure. The illustrated computer 602 is communicably coupled with a network 630. In some implementations, one or more components of the computer 602 can be configured to operate within different environments, including cloud-computing-based environments, local environments, global environments, and combinations of environments.

At a high level, the computer 602 is an electronic computing device operable to receive, transmit, process, store, and manage data and information associated with the described subject matter. According to some implementations, the computer 602 can also include, or be communicably coupled with, an application server, an email server, a web server, a caching server, a streaming data server, or a combination of servers.

The computer 602 can receive requests over network 630 from a client application (for example, executing on another computer 602). The computer 602 can respond to the received requests by processing the received requests using software applications. Requests can also be sent to the computer 602 from internal users (for example, from a command console), external (or third) parties, automated applications, entities, individuals, systems, and computers.

Each of the components of the computer 602 can communicate using a system bus 603. In some implementations, any or all of the components of the computer 602, including hardware or software components, can interface with each other or the interface 604 (or a combination of both), over the system bus 603. Interfaces can use an application programming interface (API) 612, a service layer 613, or a combination of the API 612 and service layer 613. The API 612 can include specifications for routines, data structures, and object classes. The API 612 can be either computer-language independent or dependent. The API 612 can refer to a complete interface, a single function, or a set of APIs.

The service layer 613 can provide software services to the computer 602 and other components (whether illustrated or not) that are communicably coupled to the computer 602. The functionality of the computer 602 can be accessible for all service consumers using this service layer. Software services, such as those provided by the service layer 613, can provide reusable, defined functionalities through a defined interface. For example, the interface can be software written in JAVA, C++, or a language providing data in extensible markup language (XML) format. While illustrated as an integrated component of the computer 602, in alternative implementations, the API 612 or the service layer 613 can be stand-alone components in relation to other components of the computer 602 and other components communicably coupled to the computer 602. Moreover, any or all parts of the API 612 or the service layer 613 can be implemented as child or sub-modules of another software module, enterprise application, or hardware module without departing from the scope of the present disclosure.

The computer 602 includes an interface 604. Although illustrated as a single interface 604 in FIG. 5, two or more interfaces 604 can be used according to particular needs, desires, or particular implementations of the computer 602 and the described functionality. The interface 604 can be used by the computer 602 for communicating with other systems that are connected to the network 630 (whether illustrated or not) in a distributed environment. Generally, the interface 604 can include, or be implemented using, logic encoded in software or hardware (or a combination of software and hardware) operable to communicate with the network 630. More specifically, the interface 604 can include software supporting one or more communication protocols associated with communications. As such, the network 630 or the interface's hardware can be operable to communicate physical signals within and outside of the illustrated computer 602.

The computer 602 includes a processor 605. Although illustrated as a single processor 605 in FIG. 5, two or more processors 605 can be used according to particular needs, desires, or particular implementations of the computer 602 and the described functionality. Generally, the processor 605 can execute instructions and can manipulate data to perform the operations of the computer 602, including operations using algorithms, methods, functions, processes, flows, and procedures as described in the present disclosure.

The computer 602 also includes a database 606 that can hold data for the computer 602 and other components connected to the network 630 (whether illustrated or not). For example, database 606 can be an in-memory, conventional, or a database storing data consistent with the present disclosure. In some implementations, database 606 can be a combination of two or more different database types (for example, hybrid in-memory and conventional databases) according to particular needs, desires, or particular implementations of the computer 602 and the described functionality. Although illustrated as a single database 606 in FIG. 5, two or more databases (of the same, different, or combination of types) can be used according to particular needs, desires, or particular implementations of the computer 602 and the described functionality. While database 606 is illustrated as an internal component of the computer 602, in alternative implementations, database 606 can be external to the computer 602.

The computer 602 also includes a memory 607 that can hold data for the computer 602 or a combination of components connected to the network 630 (whether illustrated or not). Memory 607 can store any data consistent with the present disclosure. In some implementations, memory 607 can be a combination of two or more different types of memory (for example, a combination of semiconductor and magnetic storage) according to particular needs, desires, or particular implementations of the computer 602 and the described functionality. Although illustrated as a single memory 607 in FIG. 5, two or more memories 607 (of the same, different, or combination of types) can be used according to particular needs, desires, or particular implementations of the computer 602 and the described functionality. While memory 607 is illustrated as an internal component of the computer 602, in alternative implementations, memory 607 can be external to the computer 602.

The application 608 can be an algorithmic software engine providing functionality according to particular needs, desires, or particular implementations of the computer 602 and the described functionality. For example, application 608 can serve as one or more components, modules, or applications. Further, although illustrated as a single application 608, the application 608 can be implemented as multiple applications 608 on the computer 602. In addition, although illustrated as internal to the computer 602, in alternative implementations, the application 608 can be external to the computer 602.

The computer 602 can also include a power supply 614. The power supply 614 can include a rechargeable or non-rechargeable battery that can be configured to be either user- or non-user-replaceable. In some implementations, the power supply 614 can include power-conversion and management circuits, including recharging, standby, and power management functionalities. In some implementations, the power-supply 614 can include a power plug to allow the computer 602 to be plugged into a wall socket or a power source to, for example, power the computer 602 or recharge a rechargeable battery.

There can be any number of computers 602 associated with, or external to, a computer system containing computer 602, with each computer 602 communicating over network 630. Further, the terms “client,” “user,” and other appropriate terminology can be used interchangeably, as appropriate, without departing from the scope of the present disclosure. Moreover, the present disclosure contemplates that many users can use one computer 602 and one user can use multiple computers 602.

Implementations of the subject matter and the functional operations described in this specification can be implemented in digital electronic circuitry, in tangibly embodied computer software or firmware, in computer hardware, including the structures disclosed in this specification and their structural equivalents, or in combinations of one or more of them. Software implementations of the described subject matter can be implemented as one or more computer programs. Each computer program can include one or more modules of computer program instructions encoded on a tangible, non-transitory, computer-readable computer-storage medium for execution by, or to control the operation of, data processing apparatus. Alternatively, or additionally, the program instructions can be encoded in/on an artificially generated propagated signal. For example, the signal can be a machine-generated electrical, optical, or electromagnetic signal that is generated to encode information for transmission to suitable receiver apparatus for execution by a data processing apparatus. The computer-storage medium can be a machine-readable storage device, a machine-readable storage substrate, a random or serial access memory device, or a combination of computer-storage mediums.

The terms “data processing apparatus,” “computer,” and “electronic computer device” (or equivalent as understood by one of ordinary skill in the art) refer to data processing hardware. For example, a data processing apparatus can encompass all kinds of apparatus, devices, and machines for processing data, including by way of example, a programmable processor, a computer, or multiple processors or computers. The apparatus can also include special purpose logic circuitry including, for example, a central processing unit (CPU), a field programmable gate array (FPGA), or an application specific integrated circuit (ASIC). In some implementations, the data processing apparatus or special purpose logic circuitry (or a combination of the data processing apparatus or special purpose logic circuitry) can be hardware- or software-based (or a combination of both hardware- and software-based). The apparatus can optionally include code that creates an execution environment for computer programs, for example, code that constitutes processor firmware, a protocol stack, a database management system, an operating system, or a combination of execution environments. The present disclosure contemplates the use of data processing apparatuses with or without conventional operating systems, for example, LINUX, UNIX, WINDOWS, MAC OS, ANDROID, or IOS.

A computer program, which can also be referred to or described as a program, software, a software application, a module, a software module, a script, or code, can be written in any form of programming language. Programming languages can include, for example, compiled languages, interpreted languages, declarative languages, or procedural languages. Programs can be deployed in any form, including as stand-alone programs, modules, components, subroutines, or units for use in a computing environment. A computer program can, but need not, correspond to a file in a file system. A program can be stored in a portion of a file that holds other programs or data, for example, one or more scripts stored in a markup language document, in a single file dedicated to the program in question, or in multiple coordinated files storing one or more modules, sub programs, or portions of code. A computer program can be deployed for execution on one computer or on multiple computers that are located, for example, at one site or distributed across multiple sites that are interconnected by a communication network. While portions of the programs illustrated in the various figures may be shown as individual modules that implement the various features and functionality through various objects, methods, or processes, the programs can instead include a number of sub-modules, third-party services, components, and libraries. Conversely, the features and functionality of various components can be combined into single components as appropriate. Thresholds used to make computational determinations can be statically, dynamically, or both statically and dynamically determined.

The methods, processes, or logic flows described in this specification can be performed by one or more programmable computers executing one or more computer programs to perform functions by operating on input data and generating output. The methods, processes, or logic flows can also be performed by, and apparatus can also be implemented as, special purpose logic circuitry, for example, a CPU, an FPGA, or an ASIC.

Computers suitable for the execution of a computer program can be based on one or more of general and special purpose microprocessors and other kinds of CPUs. The elements of a computer are a CPU for performing or executing instructions and one or more memory devices for storing instructions and data. Generally, a CPU can receive instructions and data from (and write data to) a memory. A computer can also include, or be operatively coupled to, one or more mass storage devices for storing data. In some implementations, a computer can receive data from, and transfer data to, the mass storage devices including, for example, magnetic, magneto optical disks, or optical disks. Moreover, a computer can be embedded in another device, for example, a mobile telephone, a personal digital assistant (PDA), a mobile audio or video player, a game console, a global positioning system (GPS) receiver, or a portable storage device such as a universal serial bus (USB) flash drive.

Computer readable media (transitory or non-transitory, as appropriate) suitable for storing computer program instructions and data can include all forms of permanent/non-permanent and volatile/non-volatile memory, media, and memory devices. Computer readable media can include, for example, semiconductor memory devices such as random access memory (RAM), read only memory (ROM), phase change memory (PRAM), static random access memory (SRAM), dynamic random access memory (DRAM), erasable programmable read-only memory (EPROM), electrically erasable programmable read-only memory (EEPROM), and flash memory devices. Computer readable media can also include, for example, magnetic devices such as tape, cartridges, cassettes, and internal/removable disks. Computer readable media can also include magneto optical disks and optical memory devices and technologies including, for example, digital video disc (DVD), CD ROM, DVD+/−R, DVD-RAM, DVD-ROM, HD-DVD, and BLURAY. The memory can store various objects or data, including caches, classes, frameworks, applications, modules, backup data, jobs, web pages, web page templates, data structures, database tables, repositories, and dynamic information. Types of objects and data stored in memory can include parameters, variables, algorithms, instructions, rules, constraints, and references. Additionally, the memory can include logs, policies, security or access data, and reporting files. The processor and the memory can be supplemented by, or incorporated in, special purpose logic circuitry.

Implementations of the subject matter described in the present disclosure can be implemented on a computer having a display device for providing interaction with a user, including displaying information to (and receiving input from) the user. Types of display devices can include, for example, a cathode ray tube (CRT), a liquid crystal display (LCD), a light-emitting diode (LED), and a plasma monitor. Display devices can include a keyboard and pointing devices including, for example, a mouse, a trackball, or a trackpad. User input can also be provided to the computer through the use of a touchscreen, such as a tablet computer surface with pressure sensitivity or a multi-touch screen using capacitive or electric sensing. Other kinds of devices can be used to provide for interaction with a user, including to receive user feedback including, for example, sensory feedback including visual feedback, auditory feedback, or tactile feedback. Input from the user can be received in the form of acoustic, speech, or tactile input. In addition, a computer can interact with a user by sending documents to, and receiving documents from, a device that is used by the user. For example, the computer can send web pages to a web browser on a user's client device in response to requests received from the web browser.

The term “graphical user interface,” or “GUI,” can be used in the singular or the plural to describe one or more graphical user interfaces and each of the displays of a particular graphical user interface. Therefore, a GUI can represent any graphical user interface, including, but not limited to, a web browser, a touch screen, or a command line interface (CLI) that processes information and efficiently presents the information results to the user. In general, a GUI can include a plurality of user interface (UI) elements, some or all associated with a web browser, such as interactive fields, pull-down lists, and buttons. These and other UI elements can be related to or represent the functions of the web browser.

Implementations of the subject matter described in this specification can be implemented in a computing system that includes a back-end component (for example, as a data server), or that includes a middleware component (for example, an application server). Moreover, the computing system can include a front-end component, for example, a client computer having one or both of a graphical user interface or a Web browser through which a user can interact with the computer. The components of the system can be interconnected by any form or medium of wireline or wireless digital data communication (or a combination of data communication) in a communication network. Examples of communication networks include a local area network (LAN), a radio access network (RAN), a metropolitan area network (MAN), a wide area network (WAN), Worldwide Interoperability for Microwave Access (WIMAX), a wireless local area network (WLAN) (for example, using 802.11 a/b/g/n or 802.20 or a combination of protocols), all or a portion of the Internet, or any other communication system or systems at one or more locations (or a combination of communication networks). The network can communicate with, for example, Internet Protocol (IP) packets, frame relay frames, asynchronous transfer mode (ATM) cells, voice, video, data, or a combination of communication types between network addresses.

The computing system can include clients and servers. A client and server can generally be remote from each other and can typically interact through a communication network. The relationship of client and server can arise by virtue of computer programs running on the respective computers and having a client-server relationship.

Cluster file systems can be any file system type accessible from multiple servers for read and update. Locking or consistency tracking may not be necessary since the locking of exchange file system can be done at application layer. Furthermore, Unicode data files can be different from non-Unicode data files.

### Administration of Anti-CD20 Antibodies and Fragments

The present disclosure provides methods of treating specific disorders with therapeutic compositions including an anti-CD20 antibody (e.g., Ocrevus®) or fragment thereof. The present disclosure also provides an anti-CD20 antibody (e.g., Ocrevus®) or fragment thereof for use in methods of treating specific disorders with therapeutic compositions.

The methods used herein are useful for treating a subject in need thereof. In some embodiments, disclosed herein are methods of treating a subject with an anti-CD20 antibody, the method comprising administering a therapeutically effective amount of the anti-CD20 antibody to a subject exhibiting at least one symptom of, or determined to be susceptible to, a CD20-related disorder selected from the group consisting of vascular myelopathy, ankylosing spondylitis, scleroderma, narcolepsy, gastro malabsorption conditions, complex regional pain syndrome, a cluster headache, amyloidosis, cystitis, aseptic necrosis, or combinations thereof, wherein the anti-CD20 antibody comprises: a variable heavy chain CDR1 of SEQ ID NO:1 (SYNMH); a variable heavy chain CDR2 of SEQ ID NO:2 (AIYPGNGDTSYNQKF); a variable heavy chain CDR3 of SEQ ID NO:3 (VVYYSNSYWYFDV); a variable light chain CDR1 of SEQ ID NO:4 (RASSSVSYMH); a variable light chain CDR2 of SEQ ID NO:5 (APSNLAS); and a variable light chain CDR3 of SEQ ID NO:6 (QQWSFNPPT).

In some embodiments, disclosed herein are methods of treating a subject exhibiting at least one symptom of, or determined to be susceptible to, a CD20-related disorder with an anti-CD20 antibody. In some instances, the methods include administering a therapeutically effective amount of the anti-CD20 antibody to a subject exhibiting at least one symptom of, or determined to be susceptible to, a CD20-related disorder, wherein the anti-CD20 antibody comprises: a variable heavy chain CDR1 of SEQ ID NO:1 (SYNMH); a variable heavy chain CDR2 of SEQ ID NO:2 (AIYPGNGDTSYNQKF); a variable heavy chain CDR3 of SEQ ID NO:3 (VVYYSNSYWYFDV); a variable light chain CDR1 of SEQ ID NO:4 (RASSSVSYMH); a variable light chain CDR2 of SEQ ID NO:5 (APSNLAS); and a variable light chain CDR3 of SEQ ID NO:6 (QQWSFNPPT). In some instances, the anti-CD20 antibody or antigen-binding fragment useful in the presently described methods includes three HCDRs (HCDR1, HCDR2 and HCDR3) and three LCDRs (LCDR1, LCDR2 and LCDR3), wherein the HCDR1 includes the amino acid sequence of SEQ ID NO:19 (GYTFTSYNMH); the HCDR2 includes the amino acid sequence of SEQ ID NO:20 (AIYPGNGDTS); the HCDR3 includes the amino acid sequence of SEQ ID NO:3 (VVYYSNSYWYFDV); the LCDR1 includes the amino acid sequence of SEQ ID NO:4 (RASSSVSYMH); the LCDR2 includes the amino acid sequence of SEQ ID NO:5 (APSNLAS); and the LCDR3 includes the amino acid sequence of SEQ ID NO:6 (QQWSFNPPT). In some instances, the anti-CD20 antibody or antigen-binding fragment useful anti-CD20 antibody or antigen-binding fragment useful in the presently described methods includes three HCDRs (HCDR1, HCDR2 and HCDR3) and three LCDRs (LCDR1, LCDR2 and LCDR3), wherein the HCDR1 includes the amino acid sequence of SEQ ID NO:21 (GYTFTSY); the HCDR2 includes the amino acid sequence of SEQ ID NO:22 (YPGNGD); the HCDR3 includes the amino acid sequence of SEQ ID NO:3 (VVYYSNSYWYFDV); the LCDR1 includes the amino acid sequence of SEQ ID NO:4 (RASSSVSYMH); the LCDR2 includes the amino acid sequence of SEQ ID NO:5 (APSNLAS); and the LCDR3 includes the amino acid sequence of SEQ ID NO:6 (QQWSFNPPT).

In some instances, the anti-CD20 antibody or antigen-binding fragment thereof comprises a variable heavy chain CDR1 of SEQ ID NO:1 (SYNMH); a variable heavy chain CDR2 of SEQ ID NO:2 (AIYPGNGDTSYNQKF); a variable heavy chain CDR3 of SEQ ID NO:3 (VVYYSNSYWYFDV); a variable light chain CDR1 of SEQ ID NO:4 (RASSSVSYMH); a variable light chain CDR2 of SEQ ID NO:5 (APSNLAS); and a variable light chain CDR3 of SEQ ID NO:6 (QQWSFNPPT). In some instances, the anti-CD20 antibody or antigen-binding fragment thereof includes three HCDRs (HCDR1, HCDR2 and HCDR3) and three LCDRs (LCDR1, LCDR2 and LCDR3), wherein the HCDR1 includes the amino acid sequence of SEQ ID NO:19 (GYTFTSYNMH); the HCDR2 includes the amino acid sequence of SEQ ID NO:20 (AIYPGNGDTS); the HCDR3 includes the amino acid sequence of SEQ ID NO:3 (VVYYSNSYWYFDV); the LCDR1 includes the amino acid sequence of SEQ ID NO:4 (RASSSVSYMH); the LCDR2 includes the amino acid sequence of SEQ ID NO:5 (APSNLAS); and the LCDR3 includes the amino acid sequence of SEQ ID NO:6 (QQWSFNPPT). In some instances, the anti-CD20 antibody or antigen-binding fragment thereof comprises: three HCDRs (HCDR1, HCDR2 and HCDR3) and three LCDRs (LCDR1, LCDR2 and LCDR3), wherein the HCDR1 includes the amino acid sequence of SEQ ID NO:21 (GYTFTSY); the HCDR2 includes the amino acid sequence of SEQ ID NO:22 (YPGNGD); the HCDR3 includes the amino acid sequence of SEQ ID NO:3 (VVYYSNSYWYFDV); the LCDR1 includes the amino acid sequence of SEQ ID NO:4 (RASSSVSYMH); the LCDR2 includes the amino acid sequence of SEQ ID NO:5 (APSNLAS); and the LCDR3 includes the amino acid sequence of SEQ ID NO:6 (QQWSFNPPT).

In some instances, identifying the subject for treatment includes (a) selecting a characteristic or a plurality of characteristics in a set of patients; (b) clustering, by a computer system, in accordance with the characteristic or the plurality of characteristics, a subset of patients having the characteristic or the plurality of characteristics, wherein the characteristic or the plurality of characteristics are associated with at least one symptom of, or determined to be susceptible to, the CD20-related disorder; (c) identifying in the subset of patients clustered in step (b) the CD20-related disorder based on the symptoms associated with the characteristic or the plurality of characteristics related to the CD20 pathway; and (d) selecting the subject exhibiting at least one symptom of, or determined to be susceptible to, a CD20-related disorder identified in step (c).

In some instances, the methods include identifying a subject as a candidate for treatment for a CD20-related disorder with an anti-CD20 antibody comprising (a) selecting a characteristic or a plurality of characteristics related to a CD20 pathway in a set of data representing medical records of a plurality of subjects; (b) clustering, by a computer system, in accordance with the characteristic or the plurality of characteristics related to the CD20 pathway, a subset of subjects from the plurality of subjects, wherein the subset comprises the characteristic or the plurality of characteristics; (c) identifying in the subset clustered in step (b) the CD20-related disorder based on symptoms associated with the characteristic or the plurality of characteristics related to the CD20 pathway; and (d) selecting the subject having at least one symptom of, or determined to be susceptible to, a CD20-related disorder identified in step (c). In some instances, the method further comprises administering a therapeutically effective amount of the anti-CD20 antibody to the subject, wherein the anti-CD20 antibody comprises: a variable heavy chain CDR1 of SEQ ID NO:1 (SYNMH); a variable heavy chain CDR2 of SEQ ID NO:2 (AIYPGNGDTSYNQKF); a variable heavy chain CDR3 of SEQ ID NO:3 (VVYYSNSYWYFDV); a variable light chain CDR1 of SEQ ID NO:4 (RASSSVSYMH); a variable light chain CDR2 of SEQ ID NO:5 (APSNLAS); and a variable light chain CDR3 of SEQ ID NO:6 (QQWSFNPPT). In some instances, the anti-CD20 antibody or antigen-binding fragment thereof comprises three HCDRs (HCDR1, HCDR2 and HCDR3) and three LCDRs (LCDR1, LCDR2 and LCDR3), wherein the HCDR1 includes the amino acid sequence of SEQ ID NO:19 (GYTFTSYNMH); the HCDR2 includes the amino acid sequence of SEQ ID NO:20 (AIYPGNGDTS); the HCDR3 includes the amino acid sequence of SEQ ID NO:3 (VVYYSNSYWYFDV); the LCDR1 includes the amino acid sequence of SEQ ID NO:4 (RASSSVSYMH); the LCDR2 includes the amino acid sequence of SEQ ID NO:5 (APSNLAS); and the LCDR3 includes the amino acid sequence of SEQ ID NO:6 (QQWSFNPPT). In some instances, the anti-CD20 antibody or antigen-binding fragment thereof comprises: three HCDRs (HCDR1, HCDR2 and HCDR3) and three LCDRs (LCDR1, LCDR2 and LCDR3), wherein the HCDR1 includes the amino acid sequence of SEQ ID NO:21 (GYTFTSY); the HCDR2 includes the amino acid sequence of SEQ ID NO:22 (YPGNGD); the HCDR3 includes the amino acid sequence of SEQ ID NO:3 (VVYYSNSYWYFDV); the LCDR1 includes the amino acid sequence of SEQ ID NO:4 (RASSSVSYMH); the LCDR2 includes the amino acid sequence of SEQ ID NO:5 (APSNLAS); and the LCDR3 includes the amino acid sequence of SEQ ID NO:6 (QQWSFNPPT).

In some instances, the methods include identifying a subject as a candidate for treatment for a CD20-related disorder with an anti-CD20 antibody comprising (a) selecting a characteristic or a plurality of characteristics related to a CD20 pathway in a set of data representing medical records of a plurality of subjects; (b) clustering, by a computer system, in accordance with the characteristic or the plurality of characteristics related to the CD20 pathway, a subset of subjects from the plurality of subjects, wherein the subset comprises the characteristic or the plurality of characteristics; (c) identifying in the subset clustered in step (b) the CD20-related disorder based on symptoms associated with the characteristic or the plurality of characteristics related to the CD20 pathway; and (d) selecting the subject having at least one symptom of, or determined to be susceptible to, a CD20-related disorder identified in step (c). In some instances, the method further comprises administering a therapeutically effective amount of the anti-CD20 antibody to the subject, wherein the anti-CD20 antibody comprises: a variable heavy chain CDR1 of SEQ ID NO:1 (SYNMH); a variable heavy chain CDR2 of SEQ ID NO:2 (AIYPGNGDTSYNQKF); a variable heavy chain CDR3 of SEQ ID NO:3 (VVYYSNSYWYFDV); a variable light chain CDR1 of SEQ ID NO:4 (RASSSVSYMH); a variable light chain CDR2 of SEQ ID NO:5 (APSNLAS); and a variable light chain CDR3 of SEQ ID NO:6 (QQWSFNPPT).

In some instances, the anti-CD20 antibody or antigen-binding fragment thereof comprises three HCDRs (HCDR1, HCDR2 and HCDR3) and three LCDRs (LCDR1, LCDR2 and LCDR3), wherein the HCDR1 includes the amino acid sequence of SEQ ID NO:19 (GYTFTSYNMH); the HCDR2 includes the amino acid sequence of SEQ ID NO:20 (AIYPGNGDTS); the HCDR3 includes the amino acid sequence of SEQ ID NO:3 (VVYYSNSYWYFDV); the LCDR1 includes the amino acid sequence of SEQ ID NO:4 (RASSSVSYMH); the LCDR2 includes the amino acid sequence of SEQ ID NO:5 (APSNLAS); and the LCDR3 includes the amino acid sequence of SEQ ID NO:6 (QQWSFNPPT). In some instances, the anti-CD20 antibody or antigen-binding fragment thereof comprises three HCDRs (HCDR1, HCDR2 and HCDR3) and three LCDRs (LCDR1, LCDR2 and LCDR3), wherein the HCDR1 includes the amino acid sequence of SEQ ID NO:21 (GYTFTSY); the HCDR2 includes the amino acid sequence of SEQ ID NO:22 (YPGNGD); the HCDR3 includes the amino acid sequence of SEQ ID NO:3 (VVYYSNSYWYFDV); the LCDR1 includes the amino acid sequence of SEQ ID NO:4 (RASSSVSYMH); the LCDR2 includes the amino acid sequence of SEQ ID NO:5 (APSNLAS); and the LCDR3 includes the amino acid sequence of SEQ ID NO:6 (QQWSFNPPT).

In some instances, disclosed is a method of treating a subject with an anti-CD20 antibody, comprising: (a) selecting a characteristic or a plurality of characteristics in a set of patients; (b) clustering, by a computer system, in accordance with the characteristic or the plurality of characteristics, a subset of patients having the characteristic or the plurality of characteristics, wherein the characteristic or the plurality of characteristics are associated with at least one symptom of, or determined to be susceptible to, a CD20-related disorder; and (c) administering a therapeutically effective amount of an anti-CD20 antibody to the subject exhibiting at least one symptom of, or determined to be susceptible to, the CD20-related disorder, wherein the anti-CD20 antibody comprises: a variable heavy chain CDR1 of SEQ ID NO:1 (GYTFTSYNMH); a variable heavy chain CDR2 of SEQ ID NO:2 (AIYPGNGDTSYNQKF); a variable heavy chain CDR3 of SEQ ID NO:3 (VVYYSNSYWYFDV); a variable light chain CDR1 of SEQ ID NO:4 (RASSSVSYMH); a variable light chain CDR2 of SEQ ID NO:5 (APSNLAS); and a variable light chain CDR3 of SEQ ID NO:6 (QQWSFNPPT). In some instances, the anti-CD20 antibody is an anti-CD20 antibody. In some instances, the anti-CD20 antibody is Ocrevus®. In some instances, the CD20-related disorder is a CD20-related disorder.

### I. Administration

Therapeutic compositions can be administered with suitable carriers, excipients, and other agents that are incorporated into formulations to provide improved transfer, delivery, tolerance, and the like. A multitude of appropriate formulations can be found in the formulary: Remington's Pharmaceutical Sciences, Mack Publishing Company, Easton, Pa See also Powell et al. “Compendium of excipients for parenteral formulations” PDA (1998) J Pharm Sci Technol 52:238-311, which is incorporated by reference in its entirety.

### II. Dosage

The amount of anti-CD20 antibody (e.g., Ocrevus®) administered to a subject according to the methods of the present disclosure is, generally, a therapeutically effective amount. A “therapeutically effective amount” or “therapeutically effective dosage” of a CD20 antibody is any amount of the antibody that, when used alone or in combination with another therapeutic agent, promotes disease regression evidenced by a decrease in severity of disease symptoms, an increase in frequency and duration of disease symptom-free periods, or a prevention of impairment or disability due to the disease affliction.

In some instances, the amount of anti-CD20 antibody (e.g., Ocrevus®) administered to a subject according to the methods of the present disclosure is a prophylactically effective amount. A “prophylactically effective amount” refers to an amount effective, at dosages and for periods of time necessary, to achieve the desired prophylactic result. In some aspects, since a prophylactic dose is used in subjects prior to or at an earlier stage of disease, the prophylactically effective amount is less than the therapeutically effective amount.

The dose can vary depending upon the age and the size of a subject to be administered, target disease, conditions, route of administration, and the like. When anti-CD20 antibody (e.g., Ocrevus®) is used for treating various conditions and diseases associated with CD20 in an adult patient, it is often advantageous to intravenously administer the antibody at a single dose of about 0.01 to about 20 mg/kg body weight. In some instances, the single dose is about 0.02 to about 7, about 0.03 to about 5, or about 0.05 to about 3 mg/kg body weight. Depending on the severity of the condition, the frequency and the duration of the treatment can be adjusted.

In the case of an anti-CD20 antibody (e.g., Ocrevus®), a therapeutically effective amount can be from about 0.05 mg to about 600 mg, e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg, of the anti-CD20 antibody. In certain embodiments, 300 mg of an anti-CD20 antibody is administered.

In some instances, an anti-CD20 antibody is give at an initial dose of 300 mg (e.g., in 250 mL) intravenous infusion, followed two weeks later by a second 300 mg (e.g., in 250 mL) intravenous infusion. In some instances, subsequent doses of single 600 mg (e.g., in 500 mL) intravenous infusion follow every 6 months.

The amount of an anti-CD20 antibody (e.g., Ocrevus®) contained within the individual doses can be expressed in terms of milligrams of antibody per kilogram of patient body weight (i.e., mg/kg). For example, the anti-CD20 antibody (e.g., Ocrevus®) can be administered to a patient at a dose of about 0.0001 to about 10 mg/kg of patient body weight (e.g., about 0.001 mg/kg, about 0.01 mg/kg, about 0.05 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 2.0 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg, or about 10.0 mg/kg).

In some instances, an anti-CD20 antibody (e.g., Ocrevus®) is administered at a dose of 300 mg/10 mL (30 mg/mL) in a single-dose vial.

In some instances, the subject is pre-medicated prior to administering the anti-CD20 antibody. In some instances, the subject is pre-medicated with 100 mg of methylprednisolone (or an equivalent corticosteroid), which is administered intravenously approximately 30 minutes prior to each anti-CD20 infusion. In some instances, the subject is pre-medicated with an antihistamine (e.g., diphenhydramine) approximately 30-60 minutes prior to each anti-CD20 infusion.

In some instances, one or more (e.g., 1, 2, 3, 4, or 5) doses are administered at the beginning of the treatment regimen as “loading doses” followed by subsequent doses that are administered on a less frequent basis (e.g., “maintenance doses”). For example, a CD20 antagonist may be administered to a subject at a loading dose of about 200 mg, 400 mg, or about 600 mg followed by one or more maintenance doses of about 75 mg to about 300 mg. In one embodiment, the initial dose and the one or more secondary doses each include 50 mg to 600 mg of the CD20 antagonist, e.g., 100 mg, 150 mg, 200 mg, 250 mg, 300 mg, 400 mg, 500 mg, or 600 mg of the CD20 antagonist. In some embodiments, the initial dose and the one or more secondary doses each contain the same amount of the CD20 antagonist. In other embodiments, the initial dose comprises a first amount of the CD20 antagonist, and the one or more secondary doses each comprise a second amount of the CD20 antagonist. For example, the first amount of the CD20 antagonist can be 1.5×, 2×, 2.5×, 3×, 3.5×, 4× or 5× or more than the second amount of the CD20 antagonist. In one exemplary embodiment, for a subject having a body weight that is <30 kg (e.g., ≥15 kg to <30 kg), an CD20 antagonist is administered to a subject at a loading dose of about 200 mg followed by one or more maintenance doses of about 100 mg, or at a loading dose of about 600 mg followed by one or more maintenance doses of about 300 mg. In another exemplary embodiment, for a subject having a body weight that is ≥30 kg (e.g., ≥30 kg to <60 kg), a CD20 antagonist may be administered to a subject at a loading dose of about 400 mg followed by one or more maintenance doses of about 200 mg, or at a loading dose of about 600 mg followed by one or more maintenance doses of about 300 mg. In yet another exemplary embodiment, for a subject having a body weight that is ≥60 kg, a CD20 antagonist may be administered to a subject at a loading dose of about 600 mg followed by one or more maintenance doses of about 300 mg.

In some instances, each secondary and/or tertiary dose is administered 1 to 14 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, or more) weeks after the immediately preceding dose. The phrase “the immediately preceding dose,” as used herein, means, in a sequence of multiple administrations, the dose of CD20 antagonist which is administered to a patient prior to the administration of the very next dose in the sequence with no intervening doses.

III. Delivery Various delivery systems are known and can be used to administer a pharmaceutical composition. Methods of administration include, but are not limited to, intradermal, intramuscular, intraperitoneal, intravenous, subcutaneous, intranasal, epidural, and oral routes. The composition can be administered by any convenient route, for example by infusion or bolus injection. and can be administered together with other biologically active agents. Administration can be systemic or local.

### IV. Combination Therapies

Also provided are combination therapies in which the anti-CD20 antibody (e.g., Ocrevus®) or antibody fragment is administered in combination with a second therapeutic agent. Co-administration and combination therapy are not limited to simultaneous administration, but include treatment regimens in which an anti-CD20 antibody or antibody fragment is administered at least once during a course of treatment that involves administering at least one other therapeutic agent to the patient. A second therapeutic agent can be another CD20 antagonist, such as another antibody/antibody fragment, or a soluble cytokine receptor, an IgE antagonist, an anti-asthma medication (corticosteroids, non-steroidal agents, beta agonists, leukotriene antagonists, xanthines, fluticasone, salmeterol, albuterol), or a checkpoint inhibitor, which can be delivered by inhalation or other appropriate means. In some instances, the anti-CD20 antibody or antibody fragment of the disclosure (e.g., Ocrevus®) can be administered with an IL-1 antagonist, such as rilonacept, or an IL-13 antagonist. The second agent can include one or more leukotriene receptor antagonists to treat disorders such as allergic inflammatory diseases, e.g., asthma and allergies. Examples of leukotriene receptor antagonists include but are not limited to montelukast, pranlukast, and zafirlukast. The second agent can be a checkpoint inhibitor. In some instances, the checkpoint inhibitor interferes with PD-1, interferes with PD-L1, is an anti-PD-1 antibody or is a PD-1 antagonist (e.g., a human antibody, a humanized antibody, or a chimeric antibody). In some embodiments, the anti-PD-1 antibody is MDX-1106 (also known as nivolumab, MDX-1106-04, ONO-4538, BMS-936558, and Opdivo®), Merck 3475 (pembrolizumab, MK). −3475, Rambrolizumab, Keytruda®, also known as SCH-900475), and CT-O11 (also known as Pidilizumab, hBAT, and hBAT-1). In some instances, the checkpoint inhibitor is a CTLA-4 antagonist such as Yervoy® (ipilimumab).

In some instances, the second agent can include a cytokine inhibitor such as one or more of a TNF (etanercept, ENBREL™), IL-9, IL-5 or IL-17 antagonist.

Indications Treatable with Anti-CD20 Antibodies and Fragments

### 1. Vascular Myelopathy

Vascular myelopathy is a rare severe disease caused by a broad spectrum of causes, among which pathology of the aorta and its branches, aortic surgery, spinal diseases, and spinal trauma occupy the main place. The processes of neuroinflammation and glutamate neurotoxicity play a leading role in the pathogenesis of myeloischemia. The clinical picture of the disease is nonspecific and depends on the location and volume of an ischemic focus. Magnetic resonance imaging is a gold standard for diagnosis. However, this method remains insensitive in the acute period and fails to detect spinal cord ischemia at preclinical stages. The investigation and introduction of specific biochemical markers (glutamate receptors and their antibodies) for neurotoxicity, which can identify ischemia in the advanced stage and predetermine its development, are promising.

Disclosed herein are methods of treating a subject having vascular myelopathy by administering an anti-CD20 antibody (e.g., Ocrevus®) to the subject. Also disclosed herein are methods of alleviating, ameliorating, or preventing reactions associated with vascular myelopathy by administering an anti-CD20 antibody (e.g., Ocrevus®) to the subject.

### 2. Ankylosing Spondylitis

Ankylosing spondylitis is a type of arthritis that causes inflammation in the joints and ligaments of the spine. Normally, the joints and ligaments in the spine help us move and bend. If you have ankylosing spondylitis, over time, the inflammation in the joints and tissues of the spine can cause stiffness. In some cases, this may cause the vertebrae to fuse. Symptoms associated with ankylosing spondylitis include pain, stiffness, and inflammation in other joints, such as the ribs, shoulders, knees, or feet; difficulty taking deep breaths if the joints connecting the ribs are affected; vision changes and eye pain due to uveitis, which is inflammation of the eye; fatigue, or feeling very tired; loss of appetite and weight loss; skin rashes; and abdominal pain and loose bowel movements.

Disclosed herein are methods of treating a subject having ankylosing spondylitis by administering an anti-CD20 antibody (e.g., Ocrevus®) to the subject. Also disclosed herein are methods of alleviating, ameliorating, or preventing reactions associated with ankylosing spondylitis by administering an anti-CD20 antibody (e.g., Ocrevus®) to the subject.

### 3. Scleroderma

Scleroderma is an autoimmune connective tissue and rheumatic disease that causes inflammation in the skin and other areas of the body. When an immune response tricks tissues into thinking they are injured, it causes inflammation, and the body makes too much collagen, leading to scleroderma. There are two major types of scleroderma: (1) localized scleroderma only affects the skin and the structures directly under the skin; and (2) systemic scleroderma, also called systemic sclerosis, affects many systems in the body. This is the more serious type of scleroderma and can damage your blood vessels and internal organs, such as the heart, lungs, and kidneys.

Disclosed herein are methods of treating a subject having scleroderma by administering an anti-CD20 antibody (e.g., Ocrevus®) to the subject. Also disclosed herein are methods of alleviating, ameliorating, or preventing reactions associated with scleroderma by administering an anti-CD20 antibody (e.g., Ocrevus®) to the subject.

### 4. Narcolepsy

Narcolepsy is a sleep disorder that makes people very drowsy during the day. People with narcolepsy find it hard to stay awake for long periods of time. They fall asleep suddenly. This can cause serious problems in their daily routine. Sometimes narcolepsy also causes a sudden loss of muscle tone, known as cataplexy. This can be triggered by strong emotion, especially laughter. Narcolepsy is divided into two types. Most people with type 1 narcolepsy have cataplexy. Most people who don't have cataplexy have type 2 narcolepsy. Symptoms of narcolepsy include but are not limited to excessive daytime sleepiness; sudden loss of muscle tone; sleep paralysis; hallucinations; and changes in rapid eye movement (REM) sleep.

Disclosed herein are methods of treating a subject having narcolepsy by administering an anti-CD20 antibody (e.g., Ocrevus®) to the subject. Also disclosed herein are methods of alleviating, ameliorating, or preventing reactions associated with narcolepsy by administering an anti-CD20 antibody (e.g., Ocrevus®) to the subject.

### 5. Gastro Malabsorption Conditions

Gastro malabsorption conditions include conditions having symptoms of diarrhea, steatorrhea, abdominal bloating, and gas. Other symptoms result from nutritional deficiencies. Patients often lose weight despite adequate food intake.

Some gastrointestinal diseases, such as celiac disease and inflammatory bowel disease, cause general malabsorption of all kinds of nutrients. In other cases, you may have particular difficulties absorbing a particular kind of nutrient. Some of these types include carbohydrate malabsorption, fat malabsorption, bile acid malabsorption, and protein malabsorption.

Disclosed herein are methods of treating a subject having gastro malabsorption conditions by administering an anti-CD20 antibody (e.g., Ocrevus®) to the subject. Also disclosed herein are methods of alleviating, ameliorating, or preventing reactions associated with gastro malabsorption conditions by administering an anti-CD20 antibody (e.g., Ocrevus®) to the subject.

### 6. Complex Regional Pain Syndrome

Complex regional pain syndrome (CRPS) is a broad term describing excess and prolonged pain and inflammation that follows an injury to an arm or leg. CRPS has acute and chronic (lasting greater than six months) forms. Signs and symptoms of CRPS include continuous burning or throbbing pain, usually in the arm, leg, hand or foot; sensitivity to touch or cold; swelling of the painful area; changes in skin temperature alternating between sweaty and cold; changes in skin color, ranging from white and blotchy to red or blue; changes in skin texture, which may become tender, thin or shiny in the affected area; changes in hair and nail growth; joint stiffness, swelling and damage; muscle spasms, tremors and weakness (atrophy); and decreased ability to move the affected body part.

Disclosed herein are methods of treating a subject having complex regional pain syndrome by administering an anti-CD20 antibody (e.g., Ocrevus®) to the subject. Also disclosed herein are methods of alleviating, ameliorating, or preventing reactions associated with complex regional pain syndrome by administering an anti-CD20 antibody (e.g., Ocrevus®) to the subject.

### 7. Cluster Headaches

Cluster headache is a primary headache disorder affecting up to 0.1% of the population. Patients suffer from cluster headache attacks lasting from 15 to 180 min up to 8 times a day. The attacks are characterized by the severe unilateral pain mainly in the first division of the trigeminal nerve, with associated prominent unilateral cranial autonomic symptoms and a sense of agitation and restlessness during the attacks. As compared to other disorders within the TAC category, patients with cluster headache experience multiple attacks of relatively short-lasting severe headaches. The headaches are characteristically excruciating, unilateral, and commonly involves the first division of the trigeminal nerve, over the peri- and retro-orbital regions and in the temple. The pain can be perceived to have arisen from the sinuses or from the dentition, and patients often present to an otolaryngologist or dentist for this reason. The quality of the pain is severe, intense, sharp, and burning.

Disclosed herein are methods of treating a subject having cluster headaches by administering an anti-CD20 antibody (e.g., Ocrevus®) to the subject. Also disclosed herein are methods of alleviating, ameliorating, or preventing reactions associated with cluster headaches by administering an anti-CD20 antibody (e.g., Ocrevus®) to the subject.

### 8. Amyloidosis

Amyloidosis is a rare disorder in which insoluble amyloid proteins are deposited in body organs, causing abnormal protein build-up in tissues and eventually leading to organ dysfunction and death. It affects less than 200,000 people in the United States. Definitive determination of the underlying protein is critical since prognosis and treatment of amyloidosis can vary widely depending on the responsible protein.

Disclosed herein are methods of treating a subject having amyloidosis by administering an anti-CD20 antibody (e.g., Ocrevus®) to the subject. Also disclosed herein are methods of alleviating, ameliorating, or preventing reactions associated with amyloidosis by administering an anti-CD20 antibody (e.g., Ocrevus®) to the subject.

### 9. Cystitis

Cystitis is inflammation of the bladder, usually caused by a bladder infection. It's a common type of urinary tract infection (UTI), particularly in women, and is usually more of a nuisance than a cause for serious concern. In some instances, cystitis happens when there is an infection caused by bacteria (e.g., a urinary tract infection (UTI)). In some instances, cystitis also may occur as a reaction to certain drugs or radiation therapy.

Disclosed herein are methods of treating a subject having cystitis by administering an anti-CD20 antibody (e.g., Ocrevus®) to the subject. Also disclosed herein are methods of alleviating, ameliorating, or preventing reactions associated with cystitis by administering an anti-CD20 antibody (e.g., Ocrevus®) to the subject.

### 10. Aseptic Necrosis

Aseptic necrosis is a condition in which there is a loss of blood flow to bone tissue. It is most common in the hips, knees, shoulders, and ankles. It may be caused by long-term use of steroid medicines, alcohol abuse, joint injuries, and certain diseases, such as cancer and arthritis. Disclosed herein are methods of treating a subject having aseptic necrosis by administering an anti-CD20 antibody (e.g., Ocrevus®) to the subject. Also disclosed herein are methods of alleviating, ameliorating, or preventing reactions associated with aseptic necrosis by administering an anti-CD20 antibody (e.g., Ocrevus®) to the subject.

### EXAMPLES

The disclosure is further described in the following examples, which do not limit the scope of the disclosure described in the claims.

### Example 1: Identification of Indications for Anti-CD20 Antibody

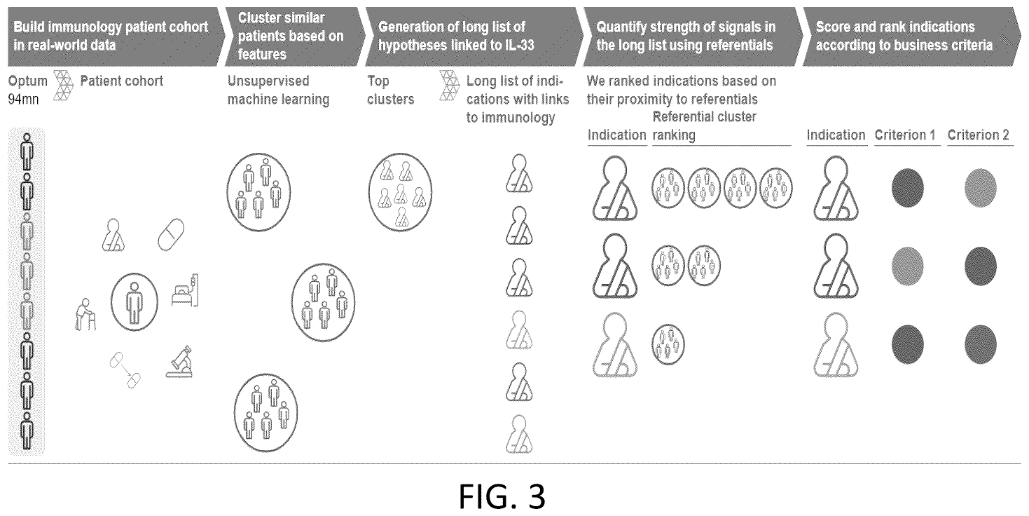

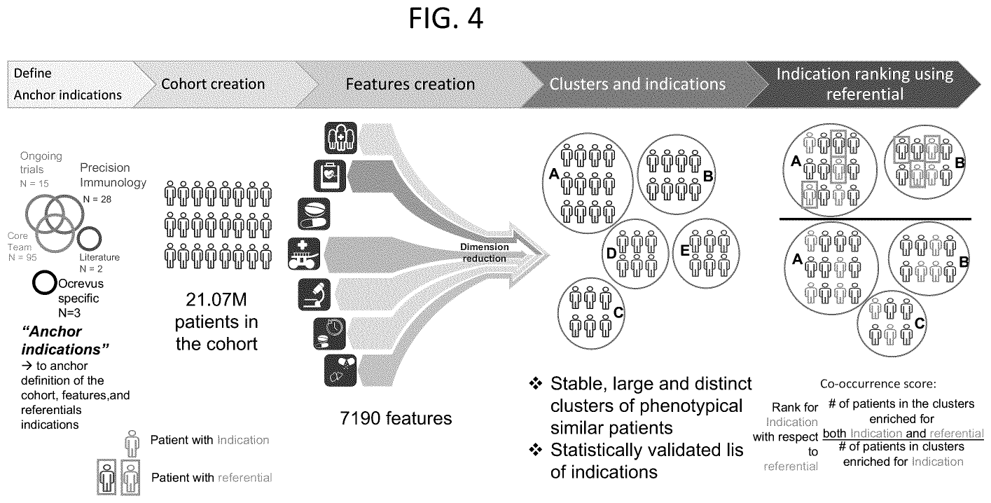

FIG. 3 is a diagram illustrating an experiment using the systems and methods described in this specification, and FIG. 4 illustrates other aspects of these methods and systems. For example, FIG. 4 illustrates how anchor indications were defined and used to identify a patient cohort, features, and referential indications; and also illustrates other aspects of feature selection, clustering, and indication ranking.

The experiment was conducted to validate a real-world data (RWD)-driven protocol for identification of indications for treatment with an anti-CD20 antibody such as Ocrevus®. A RWD-driven approach has the potential to reduce drug development cost and time to market, while minimizing attrition and risk. A hybrid approach was applied using scientific and clinical competences through KOL expertise, commercial assessment, and analytics combined with real world data.

Data Source: A real world data (RWD) deidentified dataset derived from electronic health records and electronic medical records of US patients from 2014 to 2018 was used. The dataset contained electronic health and medical records (EH/EMR) for 94 million patients identifiable by a key identifier, that allowed matching of patients across different data tables. The database collected information on EH/EMR data, such as diagnosis, lab test, procedures, medications, patient events, insurance, biomarkers, measurements, clinical status and lifestyle parameters, microbiology, and prescriptions. Natural Language Processing (NLP)-driven tables were not included, due to limited data coverage and clinical relevance. Furthermore, data tables that were incomplete or contained irrelevant information were excluded. A total of 5 data tables were included which reduced the data source to 40 million patients.

Selection of Patients: Indicators for patient selection were based on the clinical framework related ongoing trials, precision immunology, core team subjects, subjects identified in literature, and Ocrevus®-specific subjects. Anchor indications were used to identify the cohort, features, and referential indications (see FIG. 4).

Of the 21 million patients initially included, 179,565 had multiple sclerosis. 67% of these MS subjects were female, and the mean age of all subjects was 54+/−13.9 years. This group had received medical coverage for about 8.17+/−3.82 years. In the entire 21 million subject cohort, 58% of these MS subjects were female, and the mean age of all subjects was 53+/−17.9 years. This group had received medical coverage for about 7.73+/−3.92 years.

Three referential indications were used. Referential-1 included subjects treated with one prescription of Ocrevus® (n=1,365 subjects). Referential-2 included subjects treated with two or more prescription of Ocrevus® (n=4,789 subjects). Referential-3 included subjects treated with an anti-CD20-antibody (e.g., rituximab, ofatumumab, or ublituximab) for indications that included non-Hodgkin's lymphoma (NHL), chronic lymphocytic leukemia (CLL), rheumatoid arthritis (RA), granulomatosis with polyangiitis (GPA), microscopic polyangiitis (MPA), pemphigus vulgaris (PV), and follicular lymphoma (682,364 subjects).

Data from adult patients (aged ≥18 years old) with at least one diagnosis, medication, lab test and procedure who ever suffered from an CD20 pathway (i.e., signaling pathway) associated diagnosis and who were active in the 5 year window 2014-2018, were used in the analysis. Using these criteria of immunology conditions and data completeness, the resulting cohort included over 21 million unique patients.

Selection of features: Broad features (patient characteristics) were selected to capture the available information in the EH/EMR dataset; then, features selected by clinical experts were prioritized and validated to ensure that all essential variables were included, and the variable values made clinical sense. When appropriate, certain features were retained while others (certain demographics) were created de novo. New features were added based on clinical input and demographics, medication, comorbidities, procedures and laboratory tests data specific to immunology. Bespoke feature classes were created and added to the analysis iteratively to increase data completeness, representativeness, and collect more information on the severity of the disease and drug response.

A robust approach was used to ensure the completeness of the features in the final database. Two validation steps, based on patient and feature mapping across the EH/EMR database and the generated table, were taken to verify that the features were generated correctly. First, to validate whether the patient features in the EH/EMR dataset mapped to our data table correctly, the percentage of patients with at least one feature family was calculated and it was determined whether this number was identical in the EH/EMR database and in our data table. Second, to validate whether the features mapped to the correct patient, ten random patients were tracked from the raw EH/EMR data to the generated dataset, to ensure identical mapping of features in the two datasets. After the feature validation had demonstrated the correct mapping, the algorithm was run on 21 million patients with 7190 features included. The features are listed below in Table 6.

TABLE 6
Patient characterization using features.

| EH/EMR | | Number of Features |
|---|---|---|
| Source Table | Features | to Describe Patients |
| Demographics | Age groups, gender, | 3 |
| | ethnicity, region | |
| Diagnoses | Started with all ICD codes | 2629 |
| | of cohort | |
| | Removed those not relevant | |
| | for drug discovery | |
| | 30,888 ICD codes collapsed | |
| | as diseases | |
| Prescriptions | Started with all drug | 470 |
| | classes of cohort | |
| | Removed too generic | |
| | commonly used | |
| Procedures | Started with all CPT and | 2461 |
| | HCPCS codes of cohort | |
| | Selected measurements and | |
| | procedures with ≥1000 | |
| | patients 5,428 codes | |
| | collapsed to harmonized | |
| | descriptions | |
| Labs | Started with all lab | 1627 |
| | tests of cohort | |
| | Removed lab tests not | |
| | relevant as a diagnosis lab | |

Clustering: A clustering technique was used to group patients together that share similar characteristics as defined by features related to the CD20 pathway. The clustering looked for similarities between patients based on their features. The generated clusters resulted in finding correlations among conditions, even if they weren't present in the same patient. Clinical inputs were embedded in various stages of the process to ensure the clinical relevance of the results. Thus, disease experts' clinical inputs assisted in the creation of clinically relevant cohorts, in the inclusion and grouping of clinically relevant features and finally in the cluster validation and assessment. Features were identified as being distinctive in clusters if they occurred more frequently than in the general population.

Multiple Correspondence Analysis (MCA) was used to reduce the dimensions of the features. Bisecting K-means was then utilized to split the data into clusters, to provide an appropriate and effective separation of patients with sufficiently ‘tight’ but stable clusters and allow a large number of clusters that exhibited immuno-relatedness to be used for the indication scoring. The clusters identified through the process were validated and assessed by clinical experts. This step facilitated the reduction in risk of non-interpretability of the clusters and ensured the absence of overlapping features between the different clusters. The clustering approach ran on 7190 features and 21 million patients (see FIG. 4).

Identification of new indications (i.e., relevant patient characteristics): Further assessment, clinical and commercial judgement were performed to obtain a short list of priority signals and identify the most clinically relevant indications across clusters based on the cluster outputs. Four methodological steps were used. Three measures were calculated for the features included in each cluster to determine the selection: distinctiveness, the number of patients presenting the feature within each cluster, and the immunology score. The distinctiveness, also called “lift score”, measured how distinctive a feature is within a cluster versus the rest of the population (e.g. if males represent 50% of the population and 75% of the cluster, then the lift score is equal to 1.5). Only the features with a lift score >1 (meaning they occurred in the cluster at a higher rate than expected compared to a broader population) and appearing in ≥10% of the patients within the cluster were considered for defining (and naming) a cluster and developing themes of clusters. In addition, each selected feature was given a score according to its type (disease, drug, laboratory test or procedure) and immunology relevance. The features scores within each cluster were summed up and normalized. The clusters were then considered as immunology-specific if they met a pre-defined threshold of the score of 50%. As a second step, the clusters were selected according to stability, purity, and the number of patients. The stability was assessed using four methods: 1. reproducing the clusters on different sizes of data, 2. changing the initializing seeds of the clusters, 3. changing the number of clusters produced and 4. applying a train-test method. For each cluster in the train set, stability is defined as the maximum proportion of patients that are also grouped together in the test set. Purity was measured by the intra-cluster variance of MCA components of patients within that cluster, resulting in homogenous and dense clusters. A cluster is included in the analysis if it has more than 50% stability and is in the highest 20% of purity. In addition, all the indications judged by subject-matter experts as relevant to the core cluster theme were considered for evaluation, regardless of the number of patients in which these features appeared (might be <10%). These new indications were then ranked based on the frequency of co-occurrence with each of the four established indications (referential) of Ocrevus® in the third step. The co-occurrence was measured by calculating the proportion of patient-weighted clusters that contain both the indication and the referential. In the last step, the final list of indications was further characterized by clinical and commercial feasibility. The clinical assessment retained the indications that showed a distinct clinical diagnosis. Based on the ranking with the CD20 referential, conditions that did not appear in the top hits were deleted as they looked to be poorly related to CD20 modulation. The commercial assessment was possible only for a subset of clinically plausible indications, where the data on forecast sales and competitor assets were available. In addition, multiple factors were also considered for the commercial assessment: the link to the CD20 pathway, whether found or not in the literature, the worldwide prevalence of the indication and the disability-adjusted life year (DALY) of the indication (per 100,000 life years).

Results: As illustrated in FIG. 4, a cohort of >21 million patients, extracted from the EH/EMR database was used to identify a grouping of 7190 features for the input data. A feature vector was created by capturing patient characteristics in the database along with clinical input. Dimensionality reduction of the feature vector was accomplished using Multiple Correspondence Analysis (MCA), which was then used to identify distinct clusters of phenotypically similar patients. The cohort of 21 million patients was analyzed to assess data completeness and representativeness across the selected features.

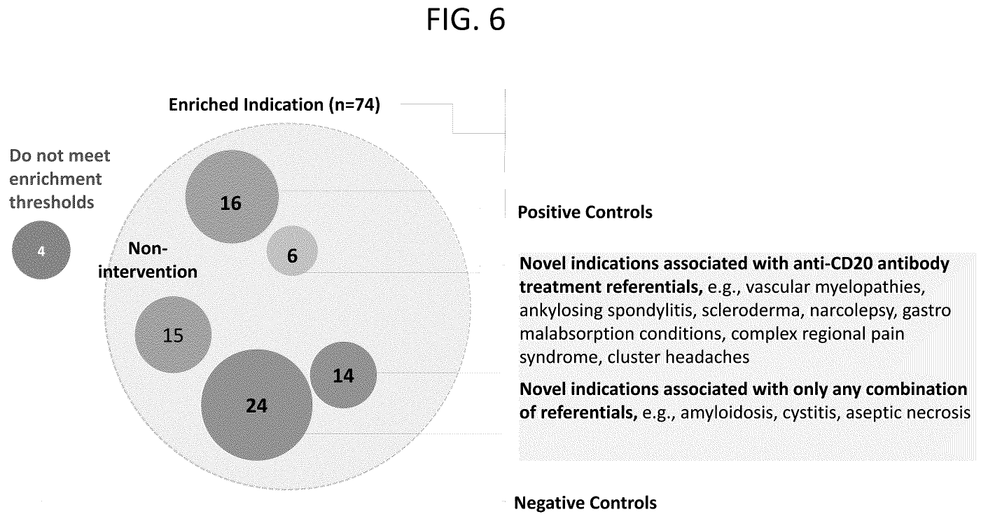

As shown in FIG. 6, there were a total of 79 enriched indications. Of these 79, four were used as a negative control. These negative control indications (including systemic lupus erythematosus (SLE), lupus nephritis) were captured in this analysis and tested for Ocre or Rituximab and failed to meet efficacy endpoints. 22 of the 79 enriched indications were identified as positive controls. Of these positive controls, 16 related to approved indications for treatment with an anti-CD20 antibody or demonstrated transient effects. These positive control indications included lymphoid leukemias, lymphomas, and anemia.

Interestingly, of the 79 enriched indications, 38 were newly identified as an enriched indication and thus suggest that treatment with an anti-CD20 antibody (e.g., Ocrevus®) would be beneficial to the subject. Among these 38 newly identified indications were vascular myelopathies, ankylosing spondylitis, scleroderma, narcolepsy, gastro malabsorption conditions, complex regional pain syndrome, cluster headaches, amyloidosis, cystitis, and aseptic necrosis. See FIG. 6.

These studies demonstrate additional indications that are suitable for treatment with an anti-CD20 antibody, such as Ocrevus®.

### Other Aspects

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

What is claimed is:

1. A method of treating a subject with an anti-CD20 antibody, the method comprising:
administering a therapeutically effective amount of the anti-CD20 antibody to a subject exhibiting at least one symptom of, or determined to be susceptible to, a CD20-related disorder selected from the group consisting of vascular myelopathy, ankylosing spondylitis, scleroderma, narcolepsy, gastro malabsorption conditions, complex regional pain syndrome, a cluster headache, amyloidosis, cystitis, aseptic necrosis, or combinations thereof,
wherein the anti-CD20 antibody comprises:
a variable heavy chain CDR1 of SEQ ID NO:1 (SYNMH);
a variable heavy chain CDR2 of SEQ ID NO:2 (AIYPGNGDTSYNQKF);
a variable heavy chain CDR3 of SEQ ID NO:3 (VVYYSNSYWYFDV);
a variable light chain CDR1 of SEQ ID NO:4 (RASSSVSYMH);
a variable light chain CDR2 of SEQ ID NO:5 (APSNLAS); and
a variable light chain CDR3 of SEQ ID NO:6 (QQWSFNPPT), each according to Kabat CDR numbering.

2. A method of treating a subject exhibiting at least one symptom of, or determined to be susceptible to, a CD20-related disorder with an anti-CD20 antibody, the method comprising:
(a) clustering, by a computer system, a subset of patients from a plurality of patients, wherein the subset of patients exhibits a characteristic or a plurality of characteristics related to a CD20 pathway;
(b) identifying in the subset of patients the CD20-related disorder based on one or more symptoms associated with the characteristic or plurality of characteristics related to the CD20 pathway; and
(c) administering a therapeutically effective amount of the anti-CD20 antibody to the subject identified in the subset of step (b),
wherein the anti-CD20 antibody comprises:
a variable heavy chain CDR1 of SEQ ID NO:1 (SYNMH);
a variable heavy chain CDR2 of SEQ ID NO:2 (AIYPGNGDTSYNQKF);
a variable heavy chain CDR3 of SEQ ID NO:3 (VVYYSNSYWYFDV);
a variable light chain CDR1 of SEQ ID NO:4 (RASSSVSYMH);
a variable light chain CDR2 of SEQ ID NO:5 (APSNLAS); and
a variable light chain CDR3 of SEQ ID NO:6 (QQWSFNPPT),
thereby treating the subject.

3. The method of claim 2, wherein the characteristic or plurality of characteristics is from a patient or a plurality of patients that has received treatment of at least one dose of the anti-CD20 antibody.

4. The method of claim 2, wherein the characteristic or plurality of characteristics is from a patient or a plurality of patients that has received treatment of two or more doses of the anti-CD20 antibody.

5. The method of claim 2, wherein the characteristic or plurality of characteristics is from a patient or a plurality of patients that has received prior treatment of at least one dose of rituximab, ofatumumab, or ublituximab.

6. The method of claim 2, wherein the CD20-related disorder comprises multiple sclerosis.

7. The method of claim 6, wherein the multiple sclerosis is primary progressive multiple sclerosis.

8. The method of claim 6, wherein the multiple sclerosis is relapsing remitting multiple sclerosis.

9. The method of claim 2, wherein the subset of patients has been diagnosed with non-Hodgkin's lymphoma, chronic lymphocytic leukemia, rheumatoid arthritis, granulomatosis with polyangiitis, microscopic polyangiitis, pemphigus vulgaris, follicular lymphoma, amyloidosis, cystitis, aseptic necrosis, or combinations thereof.

10. The method of claim 2, wherein the CD20-related disorder is selected from a vascular myelopathy, ankylosing spondylitis, scleroderma, narcolepsy, gastro malabsorption conditions, complex regional pain syndrome, a cluster headache, or combinations thereof.

11. The method of claim 2, wherein the clustering step is performed at least two, at least three, at least four or more times.

12. The method of claim 1, wherein the anti-CD20 antibody comprises:
(a)
a heavy chain variable region (VH) comprising
the amino acid sequence selected
from:
(SEQ ID NO: 7)
QAYLQQSGAELVRPGASVKMSCKASGYTFTSYNMHWVKQTPRQGL
EWIGAIYPGNGDTSYNQKFKGKATLTVDKSSSTAYMQLSSLTSED
SAVYFCARVVYYSNSYWYFDVWGTGTTVTVSS;
or
(SEQ ID NO: 8)
EVQLVESGGGLVQPGGSLRLSCAASGYTFTSYNMHWVRQAPGKGL
EWVGAIYPGNGDTSYNQKFKGRFTISVDKSKNTLYLQMNSLRAED
TAVYYCARVVYYSNSYWYFDVWGQGTLVTVSS;
and
(b)
a light chain variable region (VL) comprising
the amino acid sequence selected from:
(SEQ ID NO: 9)
QIVLSQSPAILSASPGEKVTMTCRASSSVSYMHWYQQKPGSSPKP
WIYAPSNLASGVPARFSGSGSGTSYSLTISRVEAEDAATYYCQQW
SFNPPTFGAGTKLELKR;
or
(SEQ ID NO: 10)
DIQMTQSPSSLSASVGDRVTITCRASSSVSYMHWYQQKPGKAPKP
LIYAPSNLASGVPSRFSGSGSGTDFTLTISSLQPEDFATYYCQQW
SFNPPTFGQGTKVEIKR.

13. The method of claim 1, wherein the anti-CD20 antibody comprises:
(a)
a heavy chain sequence comprising:
(SEQ ID NO: 11)
EVQLVESGGGLVQPGGSLRLSCAASGYTFTSYNMHWVRQAPGKGL
EWVGAIYPGNGDTSYNQKFKGRFTISVDKSKNTLYLQMNSLRAED
TAVYYCARVVYYSNSYWYFDVWGQGTLVTVSSASTKGPSVFPLAP
SSKSTSGGTAALGCLVKDYFPEPVTVSWNSGALTSGVHTFPAVLQ
SSGLYSLSSVVTVPSSSLGTQTYICNVNHKPSNTKVDKKVEPKSC
DKTHTCPPCPAPELLGGPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
EEMTKNQVSLTCLVKGFYPSDIAVEWESNGQPENNYKTTPPVLDS
DGSFFLYSKLTVDKSRWQQGNVFSCSVMHEALHNHYTQKSLSLSP
GK;
and
(b)
a light chain sequence comprising:
(SEQ ID NO: 12)
DIQMTQSPSSLSASVGDRVTITCRASSSVSYMHWYQQKPGKAPKP
LIYAPSNLASGVPSRFSGSGSGTDFTLTISSLQPEDFATYYCQQW
SFNPPTFGQGTKVEIKRTVAAPSVFIFPPSDEQLKSGTASVVCLL
NNFYPREAKVQWKVDNALQSGNSQESVTEQDSKDSTYSLSSTLTL
SKADYEKHKVYACEVTHQGLSSPVTKSFNRGEC.

14. The method of claim 1, wherein the anti-CD20 antibody is in a pharmaceutical composition, wherein the pharmaceutical composition comprises a pharmaceutically acceptable carrier.

15. The method of claim 1, wherein the anti-CD20 antibody is selected from a Fab fragment, a F(ab′)2 fragment, a scFv, and a scAb.

16. The method of claim 1, wherein the subject is human.

17. The method of claim 1, wherein the administering is intradermal, intramuscular, intraperitoneal, intravenous, subcutaneous, intranasal, or epidural administration.

18. The method of claim 1, wherein the anti-CD20 antibody is administered over multiple doses.

19. The method of claim 1, wherein the anti-CD20 antibody is administered at a dose of about 0.0001 to about 10 mg/kg of subject body weight.

20. The method of claim 1, further comprising administering a second therapeutic agent selected from a second antibody or antigen binding fragment thereof, a soluble cytokine receptor, an IgE antagonist, an anti-asthma medication, or a checkpoint inhibitor.
